(19) United States
(12) Patent Application Publication
(10) Pub. No.: US 2009/0060906 A1
(43) Pub. Date: Mar. 5, 2009

(54) Anti-IL-13 antibody formulations and uses thereof

(75) Inventors: Anthony B. Barry, Winchester, MA (US); Thomas J. Crowley, Wilmington, MA (US); Daniel A. Dixon, Arlington, MA (US); Erin Christine Soley, Lowell, MA (US)

Correspondence Address: WilmerHale/Wyeth, 60 STATE STREET, BOSTON, MA, 02109, US

(73) Assignee: Wyeth, Madison, NJ (US)

(21) Appl. No.: 12/008,129

(22) Filed: Jan. 9, 2008

Related U.S. Application Data
(60) Provisional application No. 60/879,500, filed on Jan. 9, 2007.

(51) Int. Cl. A61K 39/395; A61P 11/06

(52) U.S. Cl. 424/131.1

(57) ABSTRACT

Formulations suitable for treatment of disorders associated with undesirable expression or activity of IL-13 are provided.

### CROSS REFERENCE TO RELATED APPLICATIONS

This application claims the benefit of U.S. Provisional Application No. 60/879,500, filed Jan. 9, 2007, the contents of which are herein incorporated by reference in its entirety.

### TECHNICAL FIELD

This application relates to the field of antibodies, and more particularly to storage of antibodies.

### BACKGROUND

Antibodies and proteins derived from antibodies have many applications. Use of antibodies in such applications is facilitated by storage of the antibodies in formulations that promote stability of the antibodies in a variety of conditions using relatively simple formulations. If a formulation is used for a therapeutic use, it is important that the formulation permits storage without an unacceptable loss of activity of the active components, minimizes the accumulation of undesirable products such as inactive aggregates, accommodates appropriate concentrations of active components, and does not contain components that are incompatible with therapeutic applications. Formulations that are for storage of proteins to be used for downstream processing, e.g., proteins that are to be conjugated to another entity to manufacture a therapeutic must not contain components that will interfere with the manufacturing process.

### SUMMARY

The invention relates to formulations for storage of anti-IL-13 antibodies. The formulations are useful, e.g., as pharmaceutical formulations. Accordingly, in one aspect, the invention relates to an anti-IL-13 antibody formulation that includes (a) an anti-IL-13 antibody; (b) a cryoprotectant; and (c) a buffer, such that the pH of the formulation is about 5.5 to 6.5. In some embodiments, the formulation is a liquid formulation, a lyophilized formulation, a reconstituted lyophilized formulation, or an aerosol formulation. In certain embodiments, the anti-IL-13 in the formulation is at a concentration of about 0.5 mg/ml to about 250 mg/ml, about 0.5 mg/ml to about 45 mg/ml, about 0.5 mg/ml to about 100 mg/ml, about 100 mg/ml to about 200 mg/ml, or about 50 mg/ml to about 250 mg/ml. In some embodiments of the formulation, the anti-IL-13 antibody is a humanized antibody (e.g., a partially humanized antibody or a fully humanized antibody). In some cases, the antibody is a kappa light chain construct antibody. In some embodiments, the antibody is an IgG1 antibody, an IgG2 antibody, or an IgG4 antibody. In certain embodiments, the anti-IL-13 antibody in the formulation is a monoclonal antibody. In some cases, the anti-IL-13 antibody of the formulation is an antibody described in U.S. patent application Ser. No. 11/149,309 (U.S. Patent Publ. No. 20060073148), U.S. patent application Ser. No. 11/155,843 (U.S. Patent Publ. No. 20060063228), or WO 2006/085938. In specific embodiments, the anti-IL-13 antibody is IMA-638 (see, FIG. 34) or IMA-026 (see, FIG. 35).

The cryoprotectant of the formulation can be, for example, about 2.5% to about 10% (weight/volume) sucrose or trehalose. In some cases, the cryoprotectant of the formulation is not histidine. In some embodiments, the buffer in the formulation is about 4 mM to about 60 mM histidine buffer, about 5 mM to about 25 mM succinate buffer, or about 5 mM to about 25 mM acetate buffer. The pH of the buffers of the formulation is generally between about 5.0 and 7.0. In some specific embodiments, the pH of the buffer of the formulation is 5.0, 5.5, 6.0, or 6.5. Other than the cryoprotectant and buffer, the formulations of the invention may contain other excipients. In some embodiments, the formulation includes a surfactant at a concentration of about 0% to 0.2%. In some cases, the formulation contains greater than 0% and up to about 0.2% polysorbate-20, polysorbate-40, polysorbate-60, polysorbate-65, polysorbate-80 or polysorbate-85. In specific embodiments, the formulation contains 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19% or 0.2% polysorbate-80. The formulation can also include about 0.01% to about 5% arginine. In specific embodiments, the formulation contains 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19% or 0.2%, 0.3%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2%, 2.5%, 3%, 3.5%, 4.0%, 4.5% or 5% arginine. In some embodiments, the formulation also includes about 0.001% to about 0.05% Tween 20 or Tween 80. In specific embodiments, the formulation contains 0.005%, 0.008%, 0.01%, 0.2%, 0.03%, 0.04%, or 0.05% Tween 20 or Tween 80. In certain embodiments, the formulations of the invention can contain a surfactant and arginine, arginine and Tween, or arginine, Tween, and a surfactant other than Tween. In other embodiments, the formulation may also include one or more of: about 1% to about 10% sorbitol, about 0.1% to about 2% glycine, about 5 mM to about 150 mM methionine, and about 5 mM to about 100 mM sodium chloride.

The formulation can also include a second antibody or an antigen-binding fragment thereof. For example, the second antibody may be an anti-IL-13 antibody or IL-13 binding fragment thereof, wherein the second IL-13 antibody has a different epitope specificity than the first IL-13 antibody of the formulation. Other non-limiting examples of antibodies that can be co-formulated with anti-IL-13 antibody include anti-IgE antibody or an IgE binding fragment thereof, anti-IL-4 antibody or an IL-4 binding fragment thereof, an anti-TNF-α antibody or a TNF-α binding fragment thereof, an anti-C5 antibody or complement binding fragment thereof, and anti-IL-9 antibody or an IL-9 binding fragment thereof. The formulation can also include a second therapeutically or pharmacologically active agent that is useful in treating an inflammatory disorder.

In certain embodiments of the formulation, (a) the antibody is a humanized murine anti-IL-13 antibody; (b) the cryoprotectant is about 0.02% to about 10% (weight/volume) sucrose or trehalose; and (c) the buffer is about 4 mM to about 60 mM histidine buffer. In some cases, this formulation also contains about 0.01% to about 5% arginine. In certain cases, this formulation also contains about 0.001% to about 0.05% Tween. In other cases, this formulation contains about 0.01% to about 5% arginine and about 0.001% to about 0.05% Tween. In some embodiments, the formulation further comprises one or more of: about 1% to about 10% sorbitol, about 0.1% to about 2% glycine, about 5 mM to about 150 mM methionine, and about 5 mM to about 100 mM sodium chloride. In some cases, this formulation also contains greater than 0% and up to about 0.2% a surfactant (e.g., polysorbate-20, -40, -45, -60, -65, -80, -85).

In certain embodiments of the formulation, (a) the antibody is IMA-638 or IMA-026; (b) the cryoprotectant is about 0.02% to about 10% (weight/volume) sucrose or trehalose; and (c) the buffer is about 10 mM succinate buffer, pH 6.0. In other embodiments of the formulation, (a) the antibody is IMA-638 or IMA-026 antibody; (b) the cryoprotectant is about 0.02% to about 10% (weight/volume) sucrose or trehalose; and (c) the buffer is about 10 mM acetate buffer, pH 6.0.

In another aspect, an aerosol formulation is provided that comprises, (a) an anti-IL-13 antibody; (b) about 5% to about 10% (weight/volume) sucrose or trehalose; and (c) a buffer having a pH of about 5.5 to 6.5. In some cases, this formulation also contains about 0.01% to about 5% arginine. In certain cases, this formulation also contains about 0.001% to about 0.05% Tween. In other cases, this formulation contains about 0.01% to about 5% arginine and about 0.001% to about 0.05% Tween. In some embodiments, the formulation comprises one or more of: about 1% to about 10% sorbitol, about 0.1% to about 2% glycine, about 5 mM to about 150 mM methionine, and about 5 mM to about 100 mM sodium chloride. In some cases, this formulation contains greater than 0% and up to about 0.2% a surfactant (e.g., polysorbate-20, -40, -60, -65, -80, -85). In some cases, the aerosol formulation also includes a therapeutic agent that is useful in treating asthma or chronic obstructive pulmonary disease.

In another aspect, a lyophilized formulation is provided that comprises, (a) an anti-IL-13 antibody; (b) about 5% to about 10% (weight/volume) sucrose or trehalose; and (c) a buffer having a pH of about 5.5 to 6.5. In some cases, this formulation also contains about 0.01% to about 5% arginine. In certain cases, this formulation also contains about 0.001% to about 0.05% Tween. In other cases, this formulation contains about 0.01% to about 5% arginine and about 0.001% to about 0.05% Tween. In some embodiments, the formulation comprises one or more of: about 1% to about 10% sorbitol, about 0.1% to about 2% glycine, about 5 mM to about 150 mM methionine, and about 5 mM to about 100 mM sodium chloride. In some cases, this formulation contains greater than 0% and up to about 0.2% a surfactant (e.g., polysorbate-20, -40, -60, -65, -80, -85). In some cases, the lyophilized formulation also includes a therapeutic agent that is useful in treating asthma or chronic obstructive pulmonary disease.

In certain embodiments, the integrity of the antibody is maintained after storage in the formulation for at least eighteen months at −80° C., at least twenty-four months at −80° C., at least eighteen months at −20° C., at least twenty-four months at −20° C., at least eighteen months at 2° C.-8° C., at least twenty-four months at 2° C.-8° C., at least eighteen months at 25° C., or at least twenty-four months at 25° C. In some cases, the formulation includes less than 10% high molecular weight (HMW) species after at least eighteen months at −80° C., at least twenty-four months at −80° C., at least eighteen months at −20° C., at least twenty-four months at −20° C., at least eighteen months at 2° C.-8° C., at least twenty-four months at 2° C.-8° C., at least eighteen months at 25° C., or at least twenty-four months at 25° C. The invention includes embodiments in which the HMW species are assayed using size exclusion-high performance liquid chromatography (SEC-HPLC). The invention also includes embodiments in which the formulation comprises less than 10% low molecular weight (LMW) species after at least eighteen months at −80° C., at least twenty-four months at −80° C., at least eighteen months at −20° C., at least twenty-four months at −20° C., at least eighteen months at 2° C.-8° C., at least twenty-four months at 2° C.-8° C., at least eighteen months at 25° C., or at least twenty-four months at 25° C. In certain cases, the LMW species are assayed using SEC-HPLC. In some embodiments of the formulation, upon reconstitution of the lyophilized antibody formulation, the formulation retains at least 90% of the antibody structure compared to the formulation prior to lyophilization. Antibody structure is determined, for example, by binding assay, surface charge assay, bioassay, or the ratio of HMW species to LMW species.

In another aspect, the invention relates to a pharmaceutical composition for the treatment of an IL-13-related disorder. The pharmaceutical composition includes an anti-IL-13 antibody formulation as described herein, e.g., a formulation containing a humanized antibody, and other features as described herein.

In yet another aspect, the invention relates to the manufacture of a pharmaceutical composition, the composition including an antibody formulation that includes (a) an anti-IL-13 antibody; (b) a cryoprotectant; and (c) a buffer, such that the pH of the formulation is about 5.5 to 6.5. In some cases, the anti-IL-13 antibody of the pharmaceutical composition is an antibody described in U.S. patent application Ser. No. 11/149,309 (U.S. Patent Publ. No. 20060073148), U.S. patent application Ser. No. 11/155,843 (U.S. Patent Publ. No. 20060063228), or WO 2006/085938. In specific embodiments, the anti-IL-13 antibody is IMA-638 or IMA-026. In some cases, the pharmaceutical composition also contains about 0.01% to about 5% arginine. In certain cases, the pharmaceutical composition also contains about 0.001% to about 0.05% Tween. In other cases, the pharmaceutical composition contains about 0.01% to about 5% arginine and about 0.001% to about 0.05% Tween. In some embodiments, the pharmaceutical composition comprises one or more of: about 1% to about 10% sorbitol, about 0.1% to about 2% glycine, about 5 mM to about 150 mM methionine, and about 5 mM to about 100 mM sodium chloride. In some cases, this formulation contains greater than 0% and up to about 0.2% a surfactant (e.g., polysorbate-20, -40, -60, -65, -80, -85).

In another aspect, the invention relates to a method of treating an IL-13-related disorder, the method comprising administering a pharmaceutically-effective amount of an IL-13 antibody formulation. The formulation includes (a) an anti-IL-13 antibody; (b) a cryoprotectant; and (c) a buffer, such that the pH of the formulation is about 5.5 to 6.5. In some cases, the anti-IL-13 antibody of the formulation is an antibody described in U.S. patent application Ser. No. 11/149,309 (U.S. Patent Publ. No. 20060073148), U.S. patent application Ser. No. 11/155,843 (U.S. Patent Publ. No. 20060063228), or WO 2006/085938. In specific embodiments, the anti-IL-13 antibody is IMA-638 or IMA-026. In some cases, the formulation also contains about 0.01% to about 5% arginine. In certain cases, the formulation also contains about 0.001% to about 0.05% Tween. In other cases, the formulation contains about 0.01% to about 5% arginine and about 0.001% to about 0.05% Tween. In some embodiments, the formulation comprises one or more of: about 1% to about 10% sorbitol, about 0.1% to about 2% glycine, about 5 mM to about 150 mM methionine, and about 5 mM to about 100 mM sodium chloride. In some cases, this formulation contains greater than 0% and up to about 0.2% a surfactant (e.g., polysorbate-20, -40, -60, -65, -80, -85). In some embodiments, the methods of the invention includes combination therapy. Combination therapy refers to any form of administration in combination of two or more different therapeutic compounds such that the second compound is administered while the previously-administered therapeutic compound is still effective in the body (e.g., the two compounds are simultaneously effective in the patient, which may include synergistic effects of the two compounds). The combination therapy can include an anti-IL-13 antibody molecule, coformulated with and/or coadministered with one or more additional therapeutic agents, e.g., one or more cytokine and growth factor inhibitors, immunosuppressants, anti-inflammatory agents (e.g., systemic anti-inflammatory agents), metabolic inhibitors, enzyme inhibitors, and/or cytotoxic or cytostatic agents. The IL-13 binding agent and the other therapeutic can also be administered separately.

In certain embodiments of the method, the IL-13-related disorder is an inflammatory disease. In some embodiments, the inflammatory disease is selected from the group consisting of arthritis, asthma, inflammatory bowel disease, inflammatory skin disorders, multiple sclerosis, osteoporosis, tendonitis, allergic disorders, inflammation in response to an insult to the host, sepsis, rheumatoid arthritis, osteoarthritis, irritable bowel disease, ulcerative colitis, psoriasis, systematic lupus erythematosus, and any other autoimmune disease. In certain embodiments of the method, the IL-13-related disorder is allergic asthma, non-allergic asthma, combinations of allergic and non-allergic asthma, exercise induced asthma, drug-induced asthma, occupational asthma, late-stage asthma, B-cell chronic lymphocytic leukemia (B-cell CLL), Hodgkin's disease, tissue fibrosis in schistosomiasis, autoimmune rheumatic disease, inflammatory bowel disorder, rheumatoid arthritis, conditions involving airway inflammation, eosinophilia, fibrosis and excess mucus production (e.g., cystic fibrosis and pulmonary fibrosis); atopic disorders (e.g., allergic rhinitis); inflammatory and/or autoimmune conditions of the skin (e.g., atopic dermatitis), inflammatory and/or autoimmune conditions of the gastrointestinal organs (e.g., inflammatory bowel diseases (IBD)), inflammatory and/or autoimmune conditions of the liver (e.g., cirrhosis); viral infections; scleroderma and fibrosis of other organs such as liver fibrosis, allergic conjunctivitis, eczema, urticaria, food allergies, chronic obstructive pulmonary disease (COPD), ulcerative colitis, Rous Sarcoma Virus infection, uveitis, scleroderma, or osteoporosis. In some embodiments of the method, the antibody formulation is administered by inhalation, by nebulization, or injection.

In some embodiments, an injectable syringe comprising a pre-filled solution of the formulations described herein is provided. In a specific embodiment the pre-filled syringe comprises: 100 mg/ml anti-IL-13 antibody (e.g., IMA-026, IMA-638), 10 mM histidine, 5% sucrose, 0.01% Tween-80, 40 mM NaCl, pH 6.0. In another specific embodiment, the formulation in the pre-filled syringe further comprises between about 0.1% and about 2% arginine. In some cases, the syringe is provided with an autoinjector device. In other embodiments, a device for nasal administration of the formulations described herein is provided. In some cases, a transdermal patch for administration of the formulations described herein is provided. In yet other cases, an intravenous bag for administration of the formulations described herein is provided. In specific embodiments, the intravenous bag is provided with normal saline or 5% dextrose.

In other embodiments, a kit comprising a container of the formulations described herein is provided. The kit may optionally include instructions for use. In some cases, the container in the kit is a plastic or glass vial or an injectable syringe.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### DESCRIPTION OF THE DRAWINGS

FIG. 1 is a graph depicting the results of experiments in which the percentage of HMW species in lyophilized, stored anti-IL-13 antibody formulations, reconstituted at appropriate time points, was determined using size exclusion chromatography-high performance liquid chromatography (SEC-HPLC). Percent HMW=percentage of total protein in HMW species. Samples were stored at 4° C., 25° C., and 40° C. for up to twenty-four months before reconstitution.

FIG. 2 is a graph depicting the results of experiments in which the bioactivity of a lyophilized, stored, anti-IL-13 antibody formulation, reconstituted at appropriate time points, was determined as a percentage of an anti-IL-13 antibody standard. Data are expressed as specific activity in units per milligram of protein. Samples were stored at 4° C., 25° C., and 40° C. for up to twenty-four months before reconstitution.

FIG. 3 is a graph depicting the results of experiments in which the percentage of HMW species in a 100 mg/ml liquid anti-IL-13 antibody formulation was determined using SEC-HPLC after storage at 4° C., 15° C., 25° C., and 40° C. for up to twenty-four months.

FIG. 4 is a graph depicting the results of experiments in which the percentage of LMW species in a 100 mg/ml liquid anti-IL-13 antibody formulation was determined using SEC-HPLC after storage at 4° C., 15° C., 25° C., and 40° C. for up to twenty-four months.

FIG. 5 is a graph depicting the results of experiments in which the percent binding activity of anti-IL-13 antibody in a liquid formulation was assayed after storage at 4° C., 15° C., 25° C., and 40° C. for up to six months. Binding activity is expressed as a percentage relative to a standard.

FIG. 6 is a graph depicting the results of experiments in which the bioactivity of a 100 mg/ml anti-IL-13 antibody formulation was determined as a percentage of an anti-IL-13 antibody standard. Data are expressed as specific activity in units per milligram of protein. Samples were stored at 4° C., 15° C., 25° C., and 40° C. for up to twenty-four months.

FIG. 7 is a graph depicting the results of experiments assaying the protein concentration in a liquid formulation stored at 4° C., 15° C., 25° C., and 40° C. for up to twenty-four months.

FIG. 8 is a graph of sub-ambient modulated differential scanning calorimetry (mDSC) to determine the glass transition temperature of the freeze-concentrated amorphous phase.

FIG. 9A is a reproduction of a freeze-drying microscope image of an anti-IL-13 antibody at −25° C.

FIG. 9B is a reproduction of a freeze-drying microscope image of an anti-IL-13 antibody raised from −25° C. to −15° C.

FIG. 9C is a reproduction of a freeze-drying microscope image of an anti-IL-13 antibody lowered from −15° C. to −18° C.

FIG. 9D is a reproduction of a freeze-drying microscope image of an anti-IL-13 antibody raised from −18° C. to −8° C.

FIG. 9E is a reproduction of a freeze-drying microscope image of an anti-IL-13 antibody raised from −8° C. to −4° C.

FIG. 9F is a reproduction of a freeze-drying microscope image of an anti-IL-13 antibody lowered from −4° C. to −16° C.

FIG. 10 is a graph depicting a cycle trace for an aggressive lyophilization cycle. Temperature is shown for two different antibody compositions (designated MYO-029 and IMA-638), the storage shelf (shelf, and the dew point. Pressure is shown as assayed using a capacitance manometer and a Pirani gauge.

FIG. 11 is a graph depicting a cycle trace for a control lyophilization cycle. Temperature and pressure samples are as for FIG. 10.

FIG. 12 is a graph depicting a cycle trace for an annealing lyophilization cycle. Temperature and pressure samples are as for FIG. 10.

FIG. 13 is a graph depicting the product temperature during primary drying for the aggressive lyophilization cycle, the control lyophilization, and the annealing lyophilization cycles corresponding to FIGS. 10-12, respectively.

FIG. 14 is a graph depicting the modulated differential scanning calorimetry thermogram of a control sample. Two glass transition temperatures (measured on the reversing heat flow) are observed, one initiating at 51.3° C. and one at 74.5° C.

FIG. 15 is a graph depicting the results of Fourier transform infrared spectroscopy of the three samples (control, aggressive, and annealing) in the amide I region.

FIG. 16 is a graph depicting the reconstitution time of samples as a function of time in storage. Samples are control, aggressive, and annealing, and were stored at 5° C. or 50° C.

FIG. 17 is a graph depicting the protein concentration as assayed using UV-visible light spectroscopy (A₂₈₀). Samples are as for FIG. 16.

FIG. 18 is a graph depicting solution light scattering as assayed by UV-visible light spectroscopy (A₄₂₀). Samples are as for FIG. 16.

FIG. 19 is a graph depicting the results of an assay of HMW species using SEC-HPLC. Samples are as for FIG. 16.

FIG. 20 is a graph depicting the binding affinity of the tested antibody as a function of time in storage. Samples are as for FIG. 16.

FIG. 21 is a bar graph depicting the percent recovery in IMA-638 excipient screen conducted in vials and syringes, wherein the concentration of the IMA-638 antibody was measured by UV/Vis.

FIG. 22 is a bar graph depicting the percent change in HMW species in the IMA-638 excipient screen conducted in vials and syringes, from t=0 to six weeks at 40° C.

FIG. 23 is a bar graph depicting the percent change in LMW species in the IMA-638 excipient screen conducted in vials and syringes, from t=0 to six weeks at 40° C.

FIG. 24 is a bar graph depicting the concentration of IMA-638 in formulations with or without Tween following shaking at room temperature on a gel shaker for twenty-four hours at about 200 rpm.

FIG. 25 is a bar graph depicting percent HMW species of IMA-638 in formulations with or without Tween following shaking at room temperature on a gel shaker for twenty-four hours at about 200 rpm.

FIG. 26 is a bar graph depicting the concentration of IMA-638 in formulations with or without Tween following one (FT1), three (FT3), and five (FT5) freeze-thaw cycles (freeze cycle at −80° C.; thaw cycle at 37° C.).

FIG. 27 is a bar graph depicting percent HMW species of IMA-638 in formulations with or without Tween following one (FT1), three (FT3), and five (FT5) freeze-thaw cycles (freeze cycle at −80° C.; thaw cycle at 37° C.).

FIG. 28 is a graph depicting the percent HMW species in IMA-638 liquid formulations in syringes stored at 4° C. for up to 7 months.

FIG. 29 is a graph depicting the percent HMW species in IMA-638 liquid formulations in syringes stored at 25° C. for up to 7 months.

FIG. 30 is a graph depicting the percent HMW species in IMA-638 liquid formulations in syringes stored at 40° C. for up to 7 months.

FIG. 31 is a graph depicting percent HMW species in IMA-638 liquid formulations that contain 0.01% Tween and between 0% and 2% arginine in syringes stored at 40° C. for up to twenty-eight weeks.

FIG. 32 is a graph depicting the percent HMW species of an IL-13 antibody, IMA-026, that was reconstituted after being lyophilized and stored at 4° C., 25° C., and 40° C. for up to twelve months.

FIG. 33 is a graph depicting the bioactivity of an IMA-026 antibody that was reconstituted after being lyophilized and stored at 4° C., 25° C., and 40° C. for up to twelve months.

FIG. 34 provides the amino acid sequence of the IMA-638 antibody heavy chain (SEQ ID NO:1) and light chain (SEQ ID NO:2). The last amino acid residue encoded by the heavy chain DNA sequence, Lys₄₄₈, is observed in the mature, secreted form of IMA-638 only in small quantities and is presumably removed from the bulk of the monoclonal antibody during intracellular processing by Chinese hamster ovary (CHO) cellular proteases. Therefore, the carboxy-terminus of the IMA-638 heavy chain is Gly₄₄₇. Carboxy-terminus lysine processing has been observed in recombinant and plasma-derived antibodies and does not appear to impact their function.

FIG. 35 provides the amino acid sequence of the IMA-026 antibody heavy chain (SEQ ID NO:3) and light chain (SEQ ID NO:4).

### DETAILED DESCRIPTION

Formulations that include an anti-IL-13 antibody have been identified that are suitable for storage of an anti-IL-13 antibody (a “formulation”). The integrity of antibody in the formulation is generally maintained following long-term storage as a liquid or as a lyophilized product under various conditions. For example, the integrity of the antibody is adequately maintained after exposure to a wide range of storage temperatures (e.g., −80° C. to 40° C.), shear stress (e.g., shaking) and interfacial stress (freeze-thaw cycles). Additionally, for lyophilized material, the integrity of the antibody is adequately maintained during the process of reconstitution. In addition, antibody integrity is sufficiently maintained for use as a medicament as demonstrated by relatively low accumulations of LMW species and HMW species, bioactivity in vitro, binding activity in vitro, and stability after nebulization.

### Formulations

An anti-IL-13 antibody formulation as described herein includes an anti-IL-13 antibody, a compound that can serve as a cryoprotectant, and a buffer. The pH of the formulation is generally pH 5.5-6.5. In some embodiments, a formulation is stored as a liquid. In other embodiments, a formulation is prepared as a liquid and then is dried, e.g., by lyophilization or spray-drying, prior to storage. A dried formulation can be used as a dry compound, e.g., as an aerosol or powder, or reconstituted to its original or another concentration, e.g., using water, a buffer, or other appropriate liquid. The antibody purification process is designed to permit transfer of the antibody into a formulation suitable for long-term storage as a frozen liquid and subsequently for freeze-drying (e.g., using a histidine/sucrose formulation). The formulation is lyophilized with the protein at a specific concentration. The lyophilized formulation can then be reconstituted as needed with a suitable diluent (e.g., water) to resolubilize the original formulation components to a desired concentration, generally the same or higher concentration compared to the concentration prior to lyophilization. The lyophilized formulation may be reconstituted to produce a formulation that has a concentration that differs from the original concentration (i.e., before lyophilization), depending upon the amount of water or diluent added to the lyophilate relative to the volume of liquid that was originally freeze-dried (e.g., Example 6, infra)

Suitable anti-IL-13 antibody formulations can be identified by assaying one or more parameters of antibody integrity. The assayed parameters are generally the percentage of HMW species or the percentage of LMW species. The percentage of HMW species or LMW species is determined either as a percentage of the total protein content in a formulation or as a change in the percentage increase over time (i.e., during storage). The total percentage of HMW species in an acceptable formulation is not greater than 10% HMW species after storage as a lyophilate or liquid at 2° C. to 40° C. (e.g., at 2° C. to 25° C., at 2° C. to 15° C., at 2° C. to 8° C., at about 2° C., or at about 25° C.) for at least one year or not greater than about 10% LMW species after storage as a lyophilate or liquid at 2° C. to 40° C. for at least one year. By “about” is meant ±20% of a cited numerical value. Thus, “about 20° C.” means 16° C. to 24° C. Typically, the stability profile is less than 10% HMW/LMW at 2°-8° C. for a refrigerated product, and 25° C. for a room-temperature product. HMW species or LMW species are assayed in a formulation stored as a lyophilate after the lyophilate is reconstituted. 40° C. is an accelerated condition that is generally used for testing stability and determining stability for short-term exposures to non-storage conditions, e.g., as may occur during transfer of a product during shipping.

When the assayed parameter is the percentage change in HMW species or LMW species, the percent of total protein in one or both species after storage is compared to the percent total protein in one or both species prior to storage (e.g., upon preparation of the formulation). The difference in the percentages is determined. In general, the change in the percentage of protein in HMW species or LMW species in liquid formulations is not greater than 10%, e.g., not greater than about 8%, not greater than about 7%, not greater than about 6%, not greater than about 5%, not greater than about 4%, or not greater than about 3% after storage at 2°-8° C. or 25° C. for about eighteen to twenty-four months. By “about” is meant ±20% of a cited numerical value. Thus, about 10% means 8% to 12%. Formulations stored as lyophilized product generally have less than about 5%, less than about 4%, less than about 3%, or less than about 2% HMW species or less than about 5%, less than about 4%, less than about 3%, or less than about 2% LMW species after reconstitution following storage at 2° C.-8° C. (e.g., 4° C.) for about eighteen to twenty-four months.

Formulations can be stored as a lyophilate for, e.g., at least two years, at least three years, at least four years, or at least five years. In one example, an anti-IL-13 antibody formulation contains 100 mg/ml anti-IL-13 antibody, 10 mM histidine, 5% sucrose, and has a pH of 6.0. In another example, an anti-IL-13 antibody formulation contains 100 mg/ml anti-IL-13 antibody, 10 mM histidine, 5% sucrose, 0.01% Tween 80, 2% arginine, and has a pH of 6.0. In another example, the formulation contains 0.5 mg/ml anti-IL-13 antibody, 10 mM histidine, 5% sucrose, and has a pH of 6.0. In yet another example, the formulation contains 0.5 mg/ml anti-IL-13 antibody, 10 mM histidine, 5% sucrose, 0.01% Tween 80, 2% arginine, and has a pH of 6.0.

Additional details related to components of formulations and methods of assaying the integrity of anti-IL-13 antibody in a formulation are provided infra.

### Antibodies

An anti-IL-13 antibody is a component of the formulations described herein. As used herein, unless otherwise specified, the term “antibody” includes polyclonal antibodies, monoclonal antibodies, antibody compositions with polyepitope specificities, biospecific antibodies, diabodies, single chain molecules that form part of an antibody, hybrid antibodies such as fully or partially humanized antibodies, antigen-binding antibody fragments such as Fab fragments, F(ab′)₂ fragments, and Fv fragments, and modifications of the foregoing (e.g., pegylated antibodies or antibody fragments). The anti-IL-13 antibody molecule used in the formulation, can be an effectively human, humanized, CDR-grafted, chimeric, mutated, affinity matured, deimmunized, synthetic, or otherwise in vitro-generated protein. In one embodiment, the IL-13 antibody is a humanized antibody. In one embodiment, the IL-13 antibody is not antigenic in humans and does not cause a HAMA response.

An anti-IL-13 antibody molecule can be used to modulate (e.g., inhibit) at least one IL-13-associated activity in vivo. The IL-13 antibody can be used to treat or prevent an IL-13 associated-disorder, or to ameliorate at least one symptom thereof. Exemplary IL-13 associated disorders include inflammatory disorders (e.g., lung inflammation), respiratory disorders (e.g., asthma, including allergic and non-allergic asthma, chronic obstructive pulmonary disease (COPD)), as well as conditions involving airway inflammation, eosinophilia, fibrotic disorders (e.g., cystic fibrosis, liver fibrosis, and pulmonary fibrosis), scleroderma, excess mucus production; atopic disorders (e.g., atopic dermatitis, urticaria, eczema, allergic rhinitis, and allergic enterogastritis), an IL-13 associated cancer (e.g., a leukemia, glioblastoma, or lymphoma, e.g., Hodgkin's lymphoma), gastrointestinal disorders (e.g., inflammatory bowel diseases), liver disorders (e.g., cirrhosis), and viral infections.

Antibody concentrations in formulations are generally between about 0.1 mg/ml and about 250 mg/ml, e.g., about 0.5 mg/ml and about 100 mg/ml, about 0.5 mg/ml and about 1.0 mg/ml, about 0.5 mg/ml and about 45 mg/ml, about 1 mg/ml and about 10 mg/ml, about 10 mg/ml and about 40 mg/ml, about 10 mg/ml and about 50 mg/ml, about 50 mg/ml and about 100 mg/ml, about 100 mg/ml and about 200 mg/ml, about 200 mg/ml and about 250 mg/ml anti-IL-13. In the context of ranges, “about” means -20% of the lower-cited numerical value of the range and +20% of the upper-cited numerical value of the range. In the context of ranges, e.g., about 10 mg/ml to about 100 mg/ml, this means, between 8 mg/ml to 120 mg/ml. In some cases, antibody concentrations in formulations can be, for example, between 0.1 mg/ml and 200 mg/ml, e.g., 0.5 mg/ml and 100 mg/ml, 0.5 mg/ml and 1.0 mg/ml, 0.5 mg/ml and 45 mg/ml, 1 mg/ml and 10 mg/ml, 10 mg/ml and 40 mg/ml, 10 mg/ml and 50 mg/ml, 50 mg/ml and 100 mg/ml, 100 mg/ml and 200 mg/ml anti-IL-13. Such antibody formulations can be used as therapeutic agents. Accordingly, the concentration of antibody in a formulation is sufficient to provide such dosages in a volume of the formulation that is tolerated by a subject being treated and is appropriate for the method of administration. In one non-limiting example, to supply a high dosage subcutaneously, in which the volume limitation is small (e.g., about 1 ml to 1.2 ml per injection), the concentration of antibody is generally at least 100 mg/ml or greater, e.g., 100 mg/ml to 500 mg/ml, 100 mg/ml to 250 mg/ml, or 100 mg/ml to 150 mg/ml. Such high concentrations can be achieved, for example, by reconstituting a lyophilized formulation in an appropriate volume of diluent (e.g., sterile water for injection, buffered saline). In some cases, the reconstituted formulation has a concentration of between about 100 mg/ml and 500 mg/ml (e.g., 100 mg/ml, 125 mg/ml, 150 mg/ml, 175 mg/ml, 200 mg/ml, 250 mg/ml, 275 mg/ml, 300 mg/ml, 350 mg/ml, 375 mg/ml, 400 mg/ml, 425 mg/ml, 450 mg/ml, 475 mg/ml and 500 mg/ml). For delivery via inhalation, the formulation is generally somewhat concentrated (e.g., between about 100 mg/ml and 500 mg/ml) so as to provide a sufficient dose in a limited volume of aerosol for inspiration. In some cases, low concentrations (e.g., between about 0.05 mg/ml and 1 mg/ml) are used. Methods are known in the art to adapt the dosage delivered to the method of delivery, e.g., a jet nebulizer or a metered aerosol.

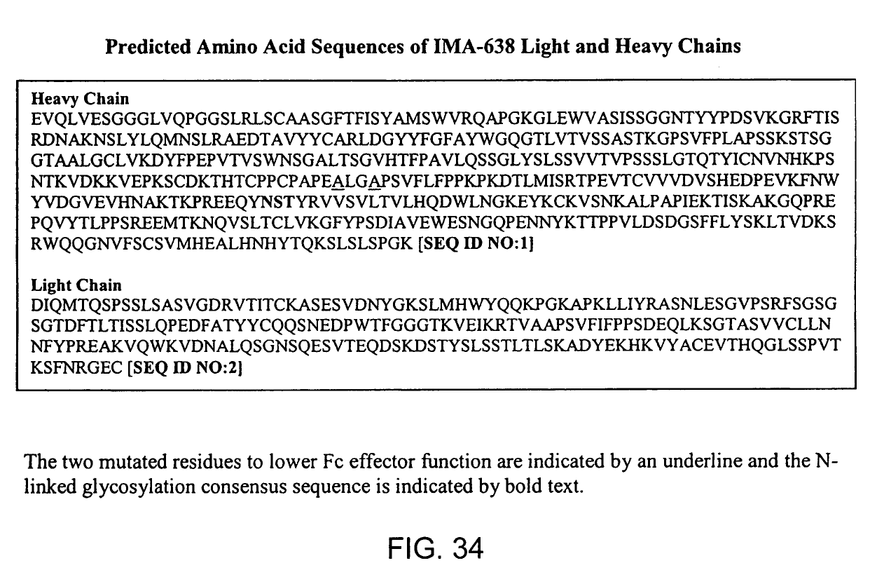

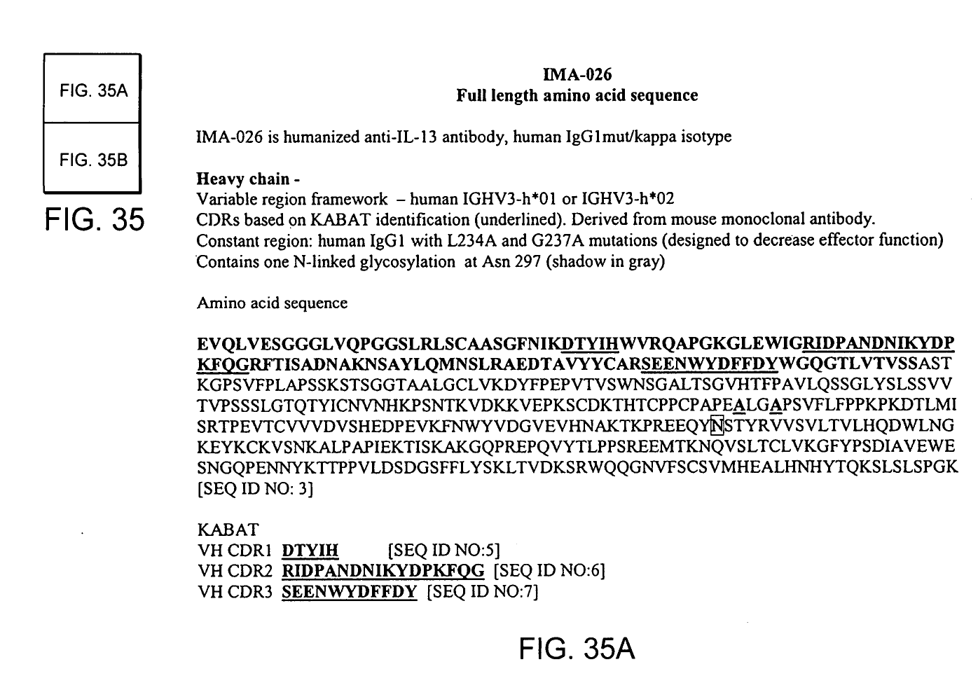

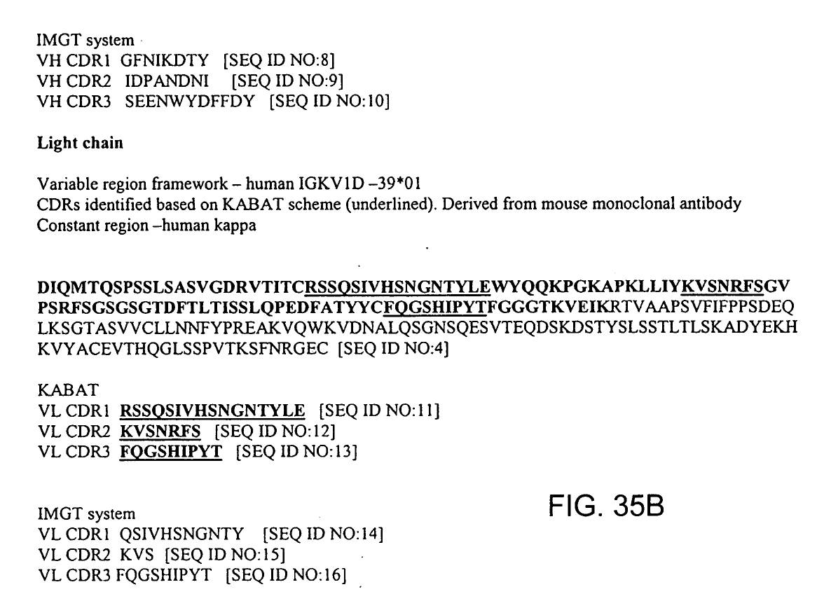

Antibodies that can be used in an anti-IL-13 antibody formulation include, e.g., murine and humanized murine anti-IL-13 antibodies. The antibodies can be kappa light chain antibodies. The antibodies can be naturally or engineered to be IgG, IgE, IgA, IgM antibodies or IL-13-binding fragments as described, supra. In some cases, the antibodies are IgG1, IgG2, or IgG4 antibodies. Examples of anti-IL-13 antibodies for use in this invention are described in U.S. patent application Ser. No. 11/155,843, U.S. patent application Ser. No. 11/149,309, and WO 2006/085938, the contents of which are herein incorporated by reference. Non-limiting examples of anti-IL-13 antibodies for use in this invention include IMA-638 (FIG. 34) and IMA-026 (FIG. 35). In some embodiments, the anti-IL-13 antibody heavy chain has about 80%, about 85%, about 90%, about 91%, about 92%, about 93%, about 94%, about 95%, about 96%, about 97%, about 98%, or about 99% sequence identity to SEQ ID NO:1 and the light chain about 80%, about 85%, about 90%, about 91%, about 92%, about 93%, about 94%, about 95%, about 96%, about 97%, about 98%, or about 99% sequence identity to SEQ ID NO:2, and the antibody binds IL-13. In some embodiments, the anti-IL-13 antibody heavy chain has about 80%, about 85%, about 90%, about 91%, about 92%, about 93%, about 94%, about 95%, about 96%, about 97%, about 98%, or about 99% sequence identity to SEQ ID NO:3 and the light chain about 80%, about 85%, about 90%, about 91%, about 92%, about 93%, about 94%, about 95%, about 96%, about 97%, about 98%, or about 99% sequence identity to SEQ ID NO:4, and the antibody binds IL-13. In certain embodiments, the anti-IL-13 antibodies bind IL-13 with an affinity corresponding to a K_{D} of less than 5×10⁻⁷ M, 1×10⁻⁷ M, 5×10⁻⁸M, 1×10⁻⁸M, 5×10⁻⁹ M, 1×10⁻⁹ M, more typically less than 5×10⁻¹⁰ M, 1×10⁻¹⁰ M, 5×10⁻¹¹ M, 1×10⁻¹¹ M, or better. Methods of introducing substitutions in a protein are well known in the art. In one embodiment, the IL-13 antibody can associate with IL-13 with kinetics in the range of 10³ to 10⁸ M⁻¹s⁻¹, typically 10⁴ to 10⁷ M⁻¹s⁻¹. In yet another embodiment, the IL-13 binding agent has dissociation kinetics in the range of 10⁻² to 10⁻⁶ s⁻¹, typically 10⁻² to 10⁻⁵ s⁻¹. In one embodiment, the IL-13 binding agent binds to IL-13, e.g., human IL-13, with an affinity and/or kinetics similar (e.g., within a factor 20, 10, or 5) to monoclonal antibody MJ 2-7 or C65 (see, U.S. Patent Publ. No. 20060073148), or modified forms thereof, e.g., chimeric forms or humanized forms thereof (e.g., a humanized form described herein). The affinity and binding kinetics of an IL-13 binding agent can be tested using, e.g., biosensor technology (BIACORE™).

### Buffers and Cryoprotectants

The pH of a formulation as described herein is generally between about pH 5.0 to about 7.0, for example, about pH 5.5 to about 6.5, about pH 5.5 to about 6.0, about pH 6.0 to about 6.5, pH 5.5, pH 6.0, or pH 6.5. In general, a buffer that can maintain a solution at pH 5.5 to 6.5 is used to prepare a formulation, e.g., a buffer having a pKA of about 6.0. Suitable buffers include, without limitation, histidine buffer, 2-morpholinoethanesulfonic acid (MES), cacodylate, phosphate, acetate, succinate, and citrate. The concentration of the buffer is between about 4 mM and about 60 mM, e.g., about 5 mM to about 25 mM, for example, histidine is generally used at a concentration of up to 60 mM. In some cases, histidine buffer is used at a concentration of about 5 mM or about 10 mM. In other cases, acetate or succinate buffer is used at a concentration of about 5 mM or about 10 mM.

An anti-IL-13 antibody formulation includes a cryoprotectant. Cryoprotectants are known in the art and include, e.g., sucrose, trehalose, and glycerol. A cryoprotectant exhibiting low toxicity in biological systems is generally used. The cryoprotectant is included in the formulation at a concentration of about 0.5% to 15%, about 0.5% to 2%, about 2% to 5%, about 5% to 10%, about 10% to 15%, and about 5% (weight/volume).

Histidine buffer, which can be used as a buffer in an anti-IL-13 antibody formulation, may have cryoprotectant properties. In some embodiments of the invention, a histidine buffer is used in conjunction with a cryoprotectant such as a sugar, e.g., sucrose. A formulation of the invention can specifically exclude the use of histidine in any substantial amount, e.g., neither the buffer nor the cryoprotectant component of the formulation is a histidine.

The viscosity of a formulation is generally one that is compatible with the route of administration of the formulation. In some embodiments, the viscosity of the formulation is between 1 cP and 2 cP, or similar to water (about 1 cP). In other embodiments, the viscosity of the formulation is between about 5 cP and about 40 cP. In specific embodiments, the viscosity of the formulation is 1 cP, 2 cP, 3 cP, 4 cP, 5 cP, 10 cP, 15 cP, 20 cP, 25 cP, 30 cP, 35 cP, or 40 cP.

### Surfactants

In certain embodiments, a surfactant is included in the formulation. Examples of surfactants include, without limitation, nonionic surfactants such as polysorbates (e.g., polysorbate-20, polysorbate-40, polysorbate-60, polysorbate-65, polysorbate-80, or polysorbate-85); poloxamers (e.g., poloxamer 188); Triton™; sodium dodecyl sulfate (SDS); sodium laurel sulfate; sodium octyl glycoside; lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauroamidopropyl-betaine, cocamidopropyl-betaine, linoleamidopropyl-betaine, myristamidopropyl-betaine, palmidopropyl-betaine, isostearamidopropyl-betaine (e.g. lauroamidopropyl), myristamidopropyl-, palmidopropyl-, or isostearamidopropyl-dimethylamine; sodium methyl cocoyl-, or disodium methyl ofeyl-taurate; and the Monaquat™ series (Mona Industries, Inc., Paterson, N.J.), polyethyl glycol, polypropyl glycol, and copolymers of ethylene and propylene glycol (e.g. pluronics, PF68).

The amount of surfactant added is such that it reduces aggregation of the reconstituted protein to an acceptable level as assayed using, e.g., SEC-HPLC of HMW species or LMW species, and minimizes the formation of particulates after reconstitution of a lyophilate of an anti-IL-13 antibody formulation. The addition of surfactant has also been shown to reduce the reconstitution time of a lyophilized formulation of anti-IL-13 antibodies, and aid in de-gassing the solution. For example, the surfactant can be present in the formulation (liquid or prior to lyophilization) in an amount from about 0.001% to 0.5%, e.g., from about 0.005% to 0.05%, about 0.005% to about 0.2%, and about 0.01% to 0.2%.

### Additions to Anti-IL-13 Formulations

Formulations are stored as sterile solutions or sterile lyophilates. Prevention of the action of microorganisms in formulations can also be achieved by including at least one antibacterial and/or antifungal agent in a formulation, for example, parabens, chlorobutanol, phenol, ascorbic acid, thimerosal, and the like. In some cases, a lyophilate is reconstituted with bacteriostatic water (e.g., water containing 0.9% benzyl alcohol). Considerations for the inclusion of a preservative in a formulation are known in the art as are methods of identifying preservatives that are compatible with a specific formulation and method of delivery (e.g., see Gupta, et al. (2003), AAPS Pharm. Sci. 5: article 8, p. 1-9).

In some cases, the formulation is isotonic. In general, any component known in the art that contributes to the solution osmolarity/tonicity can be added to a formulation (e.g., salts, sugars, polyalcohols, or a combination thereof). Isotonicity is generally achieved using either a component of a basic formulation (such as sucrose) in an isotonic concentration or by adding an additional component such as, a sugar, a polyalcohol such as manitol or sorbitol, or a salt such as sodium chloride.

In some cases, a salt is used in an anti-IL-13 antibody formulation, e.g., to achieve isotonicity or to increase the integrity of the anti-IL-13 antibody of the formulation. Salts suitable for use are discussed, supra. The salt concentration can be from 0 mM to about 300 mM.

In certain cases, the formulation is prepared with Tween (e.g., Tween® 20, Tween® 80) to decrease interfacial degradation. The Tween concentration can be from about 0.001% to about 0.05%. In one example, Tween 80 is used at a concentration of 0.01% in the formulation.

In certain other cases, the formulation is prepared with arginine. The arginine concentration in the formulation can be from about 0.01% to about 5%. In one example, arginine is used at a concentration of 2% in the formulation. In some cases both Tween and arginine are added to the IL-13 formulations described herein.

In yet other cases, the formulation may be prepared with at least one of: sorbitol, glycine, methionine, or sodium chloride. If sorbitol is included in the formulation, it can be added to a concentration of between about 1% and about 10%. In one example, sorbitol is found in the formulation at a concentration of 5%. If glycine is included in the formulation, it can be added to a concentration of between about 0.1% to about 2%. In one example, glycine is found in the formulation at a concentration of 1%. If methionine is included in the formulation, it can be added to a concentration of between about 5 mM and about 150 mM. In one example, methionine is added to the formulation at a concentration of 100 mM. In another example, methionine is added to the formulation at a concentration of 70 mM. If sodium chloride is included in the formulation, it can be added to a concentration of between about 5 mM and about 100 mM. In one example, sodium chloride is added to the formulation at a concentration of 55 mM.

### Storage and Preparation Methods

Freezing

In some cases, formulations containing antibodies are frozen for storage. Accordingly, it is desirable that the formulation be relatively stable under such conditions, including, under freeze-thaw cycles. One method of determining the suitability of a formulation is to subject a sample formulation to at least two, e.g., three, four, five, eight, ten, or more cycles of freezing (at, for example −20° C. or −80° C.) and thawing (for example by fast thaw in a 37° C. water bath or slow thaw at 2°-8° C.), determining the amount of LMW species and/or HMW species that accumulate after the freeze-thaw cycles and comparing it to the amount of LMW species or HMW species present in the sample prior to the freeze-thaw procedure. An increase in the LMW or HMW species indicates decreased stability.

Lyophilization

Formulations can be stored after lyophilization. Therefore, testing a formulation for the stability of the protein component of the formulation after lyophilization is useful for determining the suitability of a formulation. The method is similar to that described, supra, for freezing, except that the sample formulation is lyophilized instead of frozen, reconstituted to its original volume, and tested for the presence of LMW species and/or HMW species. The lyophilized sample formulation is compared to a corresponding sample formulation that was not lyophilized. An increase in LMW or HMW species in the lyophilized sample compared to the corresponding sample indicates decreased stability in the lyophilized sample. Examples of methods suitable for testing lyophilization protocols are also provided in Example 5, infra.

In general, a lyophilization protocol includes loading a sample into a lyophilizer, a pre-cooling period, freezing, vacuum initiation, ramping to the primary drying temperature, primary drying, ramping to the secondary drying temperature, secondary drying, and stoppering the sample. Additional parameters that can be selected for a lyophilization protocol include vacuum (e.g., in microns) and condenser temperature. Suitable ramp rates for temperature are between about 0.1° C./min. to 2° C./min., for example 0.1° C./min. to 1.0° C./min., 0.1° C./min. to 0.5° C./min., 0.2° C./min. to 0.5° C./min., 0.1° C./min., 0.2° C./min., 0.3° C./min., 0.4° C./min., 0.5° C./min., 0.6° C./min., 0.7° C./min., 0.8° C./min., 0.9° C./min., and 1.0° C./min. Suitable shelf temperatures during freezing for a lyophilization cycle are generally from about −55° C. to −5° C., −25° C. to −5° C., −20° C. to −5° C., −15° C. to −5° C., −10° C. to −5° C., −10° C., −11° C., −12° C., −13° C., −14° C., −15° C., −16° C., −17° C., −18° C., −19° C., −20° C., −21° C., −22° C., −23° C., −24° C., or −25° C. Shelf temperatures can be different for primary drying and secondary drying, for example, primary drying can be performed at a lower temperature than secondary drying. In a non-limiting example, primary drying can be executed at 0° C. and secondary drying at 25° C.

In some cases, an annealing protocol is used during freezing and prior to vacuum initiation. In such cases, the annealing time must be selected and the temperature is generally above the glass transition temperature of the composition. In general, the annealing time is about 2 to 15 hours, about 3 to 12 hours, about 2 to 10 hours, about 3 to 5 hours, about 3 to 4 hours, about 2 hours, about 3 hours, about 5 hours, about 8 hours, about 10 hours, about 12 hours, or about 15 hours. The temperature for annealing is generally from about −35° C. to about −5° C., for example from about −25° C. to about −8° C., about −20° C. to about −10° C., about −25° C., about −20° C., about −15° C., about 0° C., or about −5° C. In some cases, the annealing temperature is generally from −35° C. to 5° C., for example from 25° C. to −8° C., −20° C. to −10° C., −25° C., −20° C., −15° C., 0° C., or 5° C.

In one example, an anti-IL-13 antibody in a formulation described herein, was demonstrated to be robust to a variety of lyophilization parameters including: the presence or absence of a pre-vacuum thermal treatment (annealing) step above the glass transition temperature (T_{g}′), primary drying shelf temperatures from −25° C. to 30° C., and secondary drying durations of 2 hours to 9 hours at 25°-30° C.

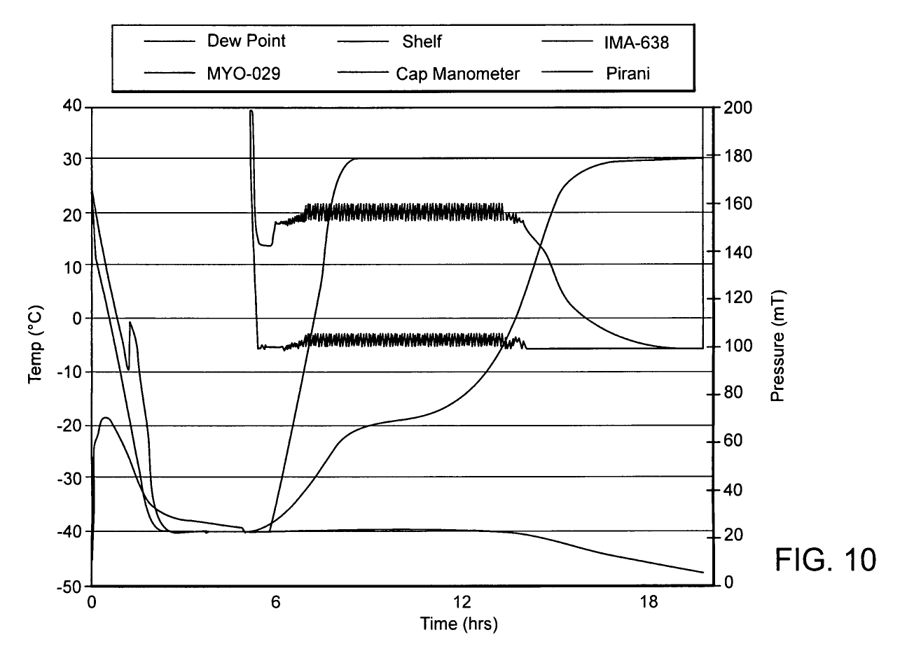

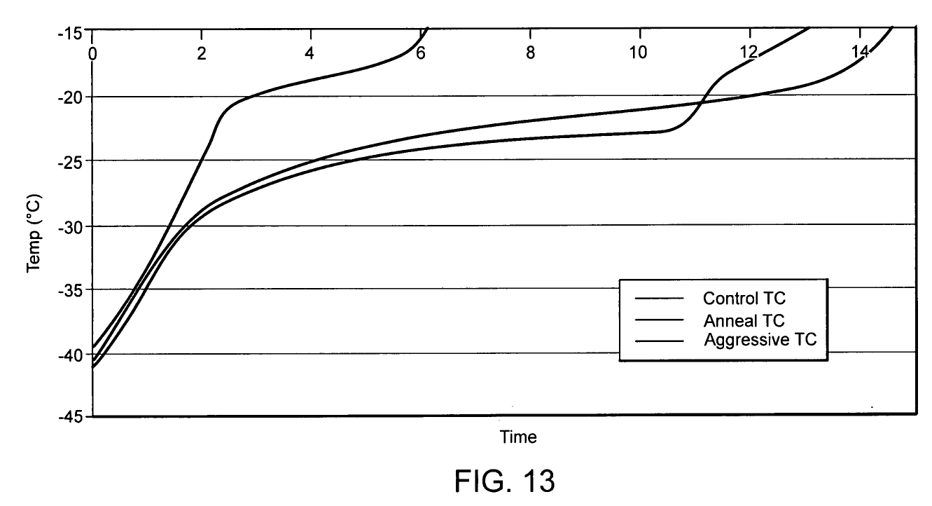

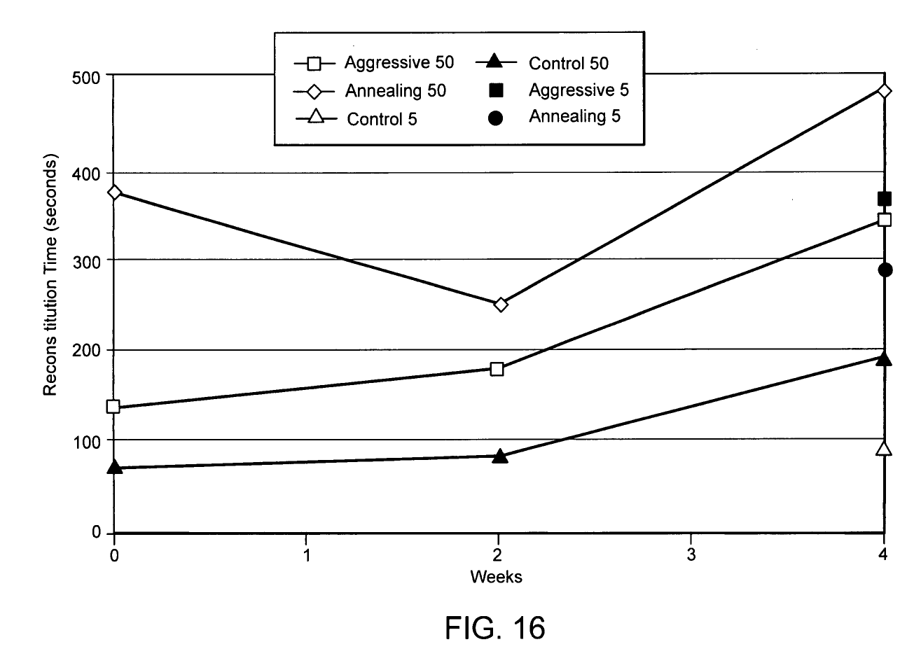

In one non-limiting example, a formulation of 10 mM histidine, 5% sucrose, pH 6.0, at a protein concentration of 50 mg/mL IL-13 was formulated in bulk and lyophilized. After lyophilization, the product is reconstituted with approximately half the fill volume to deliver protein at 100 mg/ml. The IL-13 antibody was demonstrated to be robust after lyophilization to extremes in product temperature (see Examples, infra, and FIGS. 10-12). The stability profile upon storage at 50° C. for four weeks was identical for material that had been prepared using a variety of freeze-drying cycles (e.g., see FIGS. 16-20), some of which had nearly 10° C. differences in product temperature during primary drying (e.g., FIG. 13). In general, a lyophilization cycle can run from 10 hours to 100 hours, e.g., 20 hours to 80 hours, 30 hours to 60 hours, 40 hours to 60 hours, 45 hours to 50 hours, 50 hours to 65 hours.

Non-limiting examples of the temperature range for storage of an antibody formulation are about −20° C. to about 50° C., e.g., about −15° C. to about 30° C., about −15° C. to about 20° C., about 5° C. to about 25° C., about 5° C. to about 20° C., about 5° C. to about 15° C., about 2° C. to about 12° C., about 2° C. to about 10° C., about 2° C. to about 8° C., about 2° C. to about 6° C., 2° C., 3° C., 4° C., 5° C., 6° C., 7° C., 8° C., 10° C., 15° C., or 25° C. Notwithstanding the storage temperatures, in certain cases, samples are stable under temperature changes that may transiently occur during storage and transportation conditions that can be anticipated for such compositions.

Spray-Drying

In some cases, a formulation is spray-dried and then stored. Spray-drying is conducted using methods known in the art, and can be modified to use liquid or frozen spray-drying (e.g., using methods such as those from Niro Inc. (Madison, Wis.), Upperton Particle Technologies (Nottingham, England), or Buchi (Brinkman Instruments Inc., Westbury, N.Y.), or U.S. Patent Publ. Nos. 20030072718 and 20030082276).

### Determination of Antibody Integrity

The accumulation of LMW species and HMW species are useful measures of antibody stability. Accumulation of either LMW or HMW in a formulation is indicative of instability of a protein stored as part of the formulation. Size exclusion chromatography with HPLC can be used to determine the presence of LMW and HMW species. Suitable systems for such measurements are known in the art, e.g., HPLC systems (Waters, Milford, Mass.). Other systems known in the art can be used to evaluate the integrity of antibody in a formulation, for example, SDS-PAGE (to monitor HMW and LMW species), bioassays of antibody activity, enzyme-linked immunosorbent assay, ability to bind purified IL-13 protein, and cation exchange-HPLC (CEX-HPLC; to detect variants and monitor surface charge). In one example, a bioassay is a cell-based assay in which inhibition of IL-13-dependent cell proliferation is examined in the presence of different concentrations of formulated antibody to demonstrate biological activity, i.e., the ability to bind and sequester IL-13 from the cells.

### Articles of Manufacture

The present application also provides an article of manufacture that includes a formulation as described herein and provides instructions for use of the formulation. The article of manufacture can include a container suitable for containing the formulation. A suitable container can be, without limitation, a bottle, vial, syringe, test tube, nebulizer (e.g., ultrasonic or vibrating mesh nebulizers), i.v. solution bag, or inhaler (e.g., a metered dose inhaler (MDI) or dry powder inhaler (DPI)). The container can be formed of any suitable material such as glass, metal, or a plastic such as polycarbonate, polystyrene, or polypropylene. In general, the container is of a material that does not adsorb significant amounts of protein from the formulation and is not reactive with components of the formulation. In some embodiments, the container is a clear glass vial with a West 4432/50 1319 siliconized gray stopper or a West 4023 Durafluor stopper. In some embodiments, the container is a syringe. In specific embodiments, the formulation comprises 100 mg/ml of an anti-IL-13 antibody (e.g., IMA-026, IMA-638), 10 mM histidine, 5% sucrose, 0.01% Tween-80, 40 mM NaCl, pH 6.0 in a pre-filled syringe. In certain embodiments, the syringe is suitable for use with an autoinjector device.

Examples of nebulizers include, in non-limiting examples, jet nebulizers, ultrasonic nebulizers, and vibrating mesh nebulizers. These classes use different methods to create an aerosol from a liquid. In general, any aerosol-generating device that can maintain the integrity of the protein in these formulations is suitable for delivery of formulations as described herein.

Formulations to be used for administration to a subject, e.g., as a pharmaceutical, must be sterile. This is accomplished using methods known in the art, e.g., by filtration through sterile filtration membranes, prior to, or following, formulation of a liquid or lyophilization and reconstitution. Alternatively, when it will not damage structure, components of the formulation can be sterilized by autoclaving and then combined with filter or radiation sterilized components to produce the formulation.

### Methods of Treatment

Anti-IL-13 antibody formulations are useful for treating disorders associated with undesirable expression or activity of IL-13. Such disorders include inflammatory disorders such as arthritis, asthma, inflammatory bowel disease, inflammatory skin disorders, multiple sclerosis, osteoporosis, tendonitis, allergic disorders, inflammation in response to an insult to the host, sepsis, rheumatoid arthritis, osteoarthritis, irritable bowel disease, ulcerative colitis, psoriasis, systematic lupus erythematosus, and any other autoimmune disease. In certain embodiments of the method, the IL-13-related disorder is allergic asthma, non-allergic asthma, B-cell chronic lymphocytic leukemia (B-cell CLL), Hodgkin's disease, tissue fibrosis in schistosomiasis, autoimmune rheumatic disease, inflammatory bowel disorder, rheumatoid arthritis, conditions involving airway inflammation, eosinophilia, fibrosis and excess mucus production (e.g., cystic fibrosis and pulmonary fibrosis); atopic disorders (e.g., allergic rhinitis); inflammatory and/or autoimmune conditions of the skin (e.g., atopic dermatitis), inflammatory and/or autoimmune conditions of the gastrointestinal organs (e.g., inflammatory bowel diseases (IBD)), inflammatory and/or autoimmune conditions of the liver (e.g., cirrhosis); viral infections; scleroderma and fibrosis of other organs, such as liver fibrosis, allergic conjunctivitis, eczema, urticaria, food allergies, chronic obstructive pulmonary disease (COPD), ulcerative colitis, respiratory syncytial virus infection, uveitis, scleroderma, or osteoporosis. As such, an anti-IL-13 antibody formulation can be used as a pharmaceutical composition.

The present invention provides for both prophylactic and therapeutic methods of treating a subject at risk of (or susceptible to) a disorder or having a disorder or a complication of a disorder associated with aberrant or unwanted IL-13 expression or activity. As used herein, the term “treatment” is defined as the application or administration of a therapeutic agent to a subject, or application or administration of a therapeutic agent to an isolated tissue or cell line from a subject, who has a disease, a symptom of a disease, or a predisposition toward a disease, with the purpose to cure, heal, alleviate, relieve, alter, remedy, ameliorate, improve or affect the disease, the symptoms of disease or the predisposition toward disease.

An anti-IL-13 antibody formulation can be administered to a subject in need of treatment using methods known in the art, including oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraocular, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, transdermal (topical), or transmucosal administration. For administration by inhalation, the compounds are delivered in the form of an aerosol spray from a pressured container or dispenser that contains a suitable propellant, e.g., a gas such as carbon dioxide, or a nebulizer. In certain embodiments, the formulation is administered as a sustained-release, extended-release, timed-release, controlled-release, or continuous-release formulation. In some embodiments, depot formulations are used to administer the antibody to the subject in need thereof.

Oral or parenteral compositions can be prepared in dosage unit form for ease of administration and uniformity of dosage. “Dosage unit form,” as used herein refers to physically discrete units suited as unitary dosages for the subject to be treated; each unit containing a predetermined quantity of active compound calculated to produce the desired therapeutic effect in association with the selected pharmaceutical carrier. In the case of an inhalation method such as metered dose inhaler, the device is designed to deliver an appropriate amount of the formulation.

Toxicity and therapeutic efficacy of a formulation can be determined by pharmaceutical procedures known in the art using, e.g., cell cultures or experimental animals, e.g., for determining the LD₅₀ (the dose lethal to 50% of the population) and the ED₅₀ (the dose therapeutically-effective in 50% of the population). The dose ratio between toxic and therapeutic effects is the therapeutic index, and it can be expressed as the ratio LD₅₀/ED₅₀.

The data obtained from the cell culture assays and animal studies can be used in formulating a range of dosage for use in humans. The dosage of such formulations generally lies within a range of circulating concentrations that include the ED₅₀ with little or no toxicity. The dosage may vary within this range depending upon the dosage form employed and the route of administration utilized. For any formulation used in the method of the invention, the therapeutically-effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography or specific binding assays (e.g., ELISA). Suitable animal models are known in the art and include, without limitation, non-human primates in which efficacy has been demonstrated in responding to antigen challenge, and antigen-sensitive sheep following an antigen challenge, and guinea pig.

A formulation is generally delivered such that the dosage is at least about 0.1 mg anti-IL-13 antibody/kg of body weight (generally about 1 mg/kg to about 10 mg/kg). If the antibody is to act in the brain, a dosage of 50 mg/kg to 100 mg/kg may be appropriate. The dosage may be reduced (compared to parenteral administration) when delivered directly to the site of action, for example when administered directly to lung tissue by inhalation. A formulation described herein may be used for the preparation of a medicament for use in any of the methods of treatment described herein.

### Combination Therapy

In certain aspects of the present invention, the formulations described herein can be modified so as to be administered as part of a combinatorial therapy with other agents. Combination therapy refers to any form of administration in combination of two or more different therapeutic compounds such that the second compound is administered while the previously-administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. Thus, an individual who receives such treatment can have a combined (conjoint) effect of different therapeutic compounds. Examples of preferred additional therapeutic agents that can be coadministered and/or coformulated with an IL-13 antibody include: inhaled steroids; beta-agonists, e.g., short-acting or long-acting beta-agonists; antagonists of leukotrienes or leukotriene receptors; combination drugs such as ADVAIR®; IgE inhibitors, e.g., anti-IgE antibodies (e.g., XOLAIR®); phosphodiesterase inhibitors (e.g., PDE4 inhibitors); xanthines; anticholinergic drugs; mast cell-stabilizing agents such as cromolyn; IL-4 inhibitors; IL-5 inhibitors; eotaxin/CCR3 inhibitors; and antihistamines. Such combinations can be used to treat asthma and other respiratory disorders. Additional examples of therapeutic agents that can be coadministered and/or coformulated with an IL-13 antibody include one or more of: TNF antagonists (e.g., a soluble fragment of a TNF receptor, e.g., p55 or p75 human TNF receptor or derivatives thereof, e.g., 75 kd TNFR-IgG (75 kD TNF receptor-IgG fusion protein, ENBREL™)); TNF enzyme antagonists, e.g., TNFα converting enzyme (TACE) inhibitors; muscarinic receptor antagonists; TGF-β antagonists; interferon gamma; perfenidone; chemotherapeutic agents, e.g., methotrexate, leflunomide, or a sirolimus (rapamycin) or an analog thereof, e.g., CCI-779; COX2 and cPLA2 inhibitors; NSAIDs; immunomodulators; p38 inhibitors, TPL-2, Mk-2 and NFκB inhibitors, among others.

For example, in the case of inflammatory conditions, the anti-IL-13 antibody formulations described herein can be administered in combination with one or more other agents useful in the treatment of inflammatory diseases or conditions. These agents may be formulated together with the anti-IL-13 antibody, or administered at substantially the same time as separate formulations, or sequentially. In some cases, the agent can be an IL-13 antibody that has a different epitope than the anti-IL-13 antibody of the formulation. Other agents useful in the treatment of inflammatory diseases or conditions include, but are not limited to, anti-inflammatory agents, or antiphlogistics. Antiphlogistics include, for example, glucocorticoids, such as cortisone, hydrocortisone, prednisone, prednisolone, fluorcortolone, triamcinolne, methylprednisolone, prednylidene, paramethasone, dexamethasone, betamethasone, beclomethasone, fluprednylidene, desoxymethasone, fluocinolone, flunethasone, diflucortolone, clocortolone, clobetasol and fluocortin butyl ester; immunosuppressive agents such as anti-TNF agents (e.g., etanercept, infliximab) and IL-1 inhibitors; penicillamine; non-steroidal anti-inflammatory drugs (NSAIDs) which encompass anti-inflammatory, analgesic, and antipyretic drugs such as salicyclic acid, celecoxib, difunisal and from substituted phenylacetic acid salts or 2-phenylpropionic acid salts, such as alclofenac, ibutenac, ibuprofen, clindanac, fenclorac, ketoprofen, fenoprofen, indoprofen, fenclofenac, diclofenac, flurbiprofen, piprofen, naproxen, benoxaprofen, carprofen and cicloprofen; oxican derivatives, such as piroxican; anthranilic acid derivatives, such as mefenamic acid, flufenamic acid, tolfenamic acid and meclofenamic acid, anilino-substituted nicotinic acid derivatives, such as the fenamates miflumic acid, clonixin and flunixin; heteroarylacetic acids wherein heteroaryl is a 2-indol-3-yl or pyrrol-2-yl group, such as indomethacin, oxmetacin, intrazol, acemetazin, cinmetacin, zomepirac, tolmetin, colpirac and tiaprofenic acid; idenylacetic acid of the sulindac type; analgesically-active heteroaryloxyacetic acids, such as benzadac; phenylbutazone; etodolac; nabunetone; and disease-modifying antirheumatic drugs (DMARDs) such as methotrexate, gold salts, hydroxychloroquine, sulfasalazine, ciclosporin, azathioprine, and leflunomide.

Other therapeutics useful in the treatment of inflammatory diseases or conditions include antioxidants. Antioxidants may be natural or synthetic. Antioxidants are, for example, superoxide dismutase (SOD), 21-aminosteroids/aminochromans, vitamin C or E, etc. Many other antioxidants are well known to those of skill in the art.

The anti-IL-13 antibody formulations described herein may serve as part of a treatment regimen for an inflammatory condition, which may combine many different anti-inflammatory agents. For example, the anti-IL-13 antibody formulations described herein may be administered in combination with one or more of an IL-4 inhibitor, an IL-5 inhibitor, an IgE inhibitor, an IL-9 inhibitor, a TNF antagonist, an eotaxin/CCR3 antagonist, an NSAID, a DMARD, an immunosuppressant, phosphodiesterase inhibitor, or an antihistamine. In one embodiment of the application, the anti-IL-13 antibody formulations described herein can be administered in combination with methotrexate. In another embodiment, the anti-IL-13 antibody formulations described herein can be administered in combination with a TNF-α inhibitor. In the case of asthma, the anti-IL-13 antibody formulations described herein may be administered in combination with one or more of NSAIDs, corticosteroids, leukotriene modifiers, long-acting beta-adrenergic agonists, theophylline, antihistamines, and cromolyn.

In the case of cancer, the anti-IL-13 antibody formulations described herein can be administered in combination with one or more anti-angiogenic factors, chemotherapeutics, or as an adjuvant to radiotherapy. It is further envisioned that the administration of the anti-IL-13 antibody formulations described herein will serve as part of a cancer treatment regimen which may combine many different cancer therapeutic agents. In the case of irritable bowel disease (IBD), the anti-IL-13 antibody formulations described herein can be administered with one or more anti-inflammatory agents and may additionally be combined with a modified dietary regimen.

### EXAMPLES

The invention is further illustrated by the following examples. The examples are provided for illustrative purposes only. They are not to be construed as limiting the scope or content of the invention in any way.

### Example 1

### Stability of a Lyophilized Anti-IL-13 Formulation

One method of storing an antibody to be used for, e.g., therapeutic applications, is as a dried powder prepared by lyophilization. Accordingly, the long-term stability of a lyophilized anti-IL-13 formulation was studied. Briefly, a formulation containing a humanized anti-IL-13 antibody (50 mg/ml), 10 mM histidine, 5% sucrose (weight/volume), pH 6.0, was prepared by sterile filtration and approximately 3.2 ml was dispensed into a 5 ml depyrogenated glass tubing vial, and then lyophilized. The formulation was stored at 4° C., 25° C., or 40° C. for one month, two months, three months, six months, and twelve months, as well as eighteen months and twenty-four months at 4° C. and 25° C., then reconstituted using 1.3 ml sterile water (USP) to bring the reconstituted formulation to about 1.6 ml such that the formulation was 100 mg/ml anti-IL-13 antibody, 20 mM histidine, and 10% sucrose, pH 6.0.

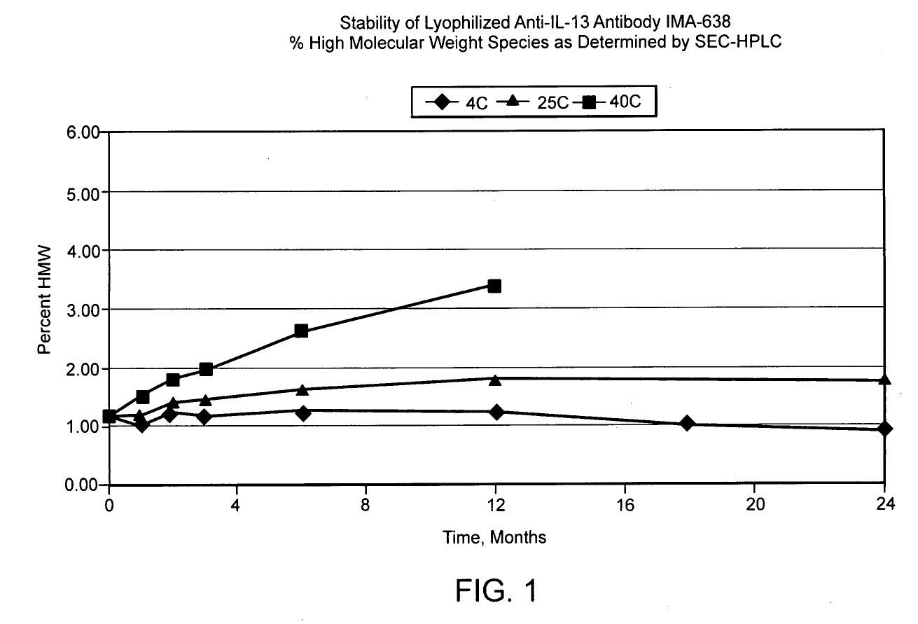

The percentage of HMW species was assayed using SEC-HPLC. The percentage of HMW species in the formulation before lyophilization and reconstitution was between 1%-1.5% of the total protein in the formulation and was also between about 1%-2% in all samples stored at 4° C. and 25° C. (FIG. 1). After twelve months of storage at 40° C., the formulations were about 3.5% HMW species (FIG. 1). Thus, there was no substantial increase in the level of HMW species in samples stored at 5° C. and 25° C. for twenty-four months.

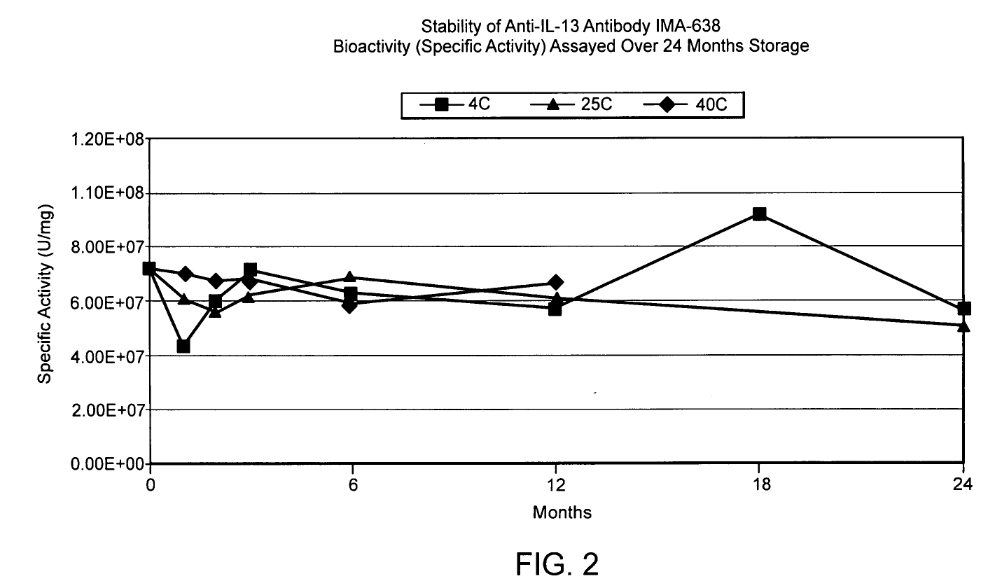

The lyophilized anti-IL-13 antibody formulations were also assayed for bioactivity using a cell-based assay in which inhibition of IL-13-dependent cell proliferation was examined in the presence of different concentrations of formulated antibody to demonstrate biological activity, i.e., the ability to bind and sequester IL-13 from the cells. The results of the assay are compared to the results using a different anti-IL-13 antibody that was not stored. FIG. 2 illustrates the data from such a set of bioassays. Overall, there was no substantial change in the amount of bioactivity after twenty-four months of storage in any of the samples. Thus, the formulation is, as determined by bioactivity, suitable for storage of the lyophilized formulation for at least twenty-four months.

These data demonstrate that a lyophilized anti-IL-13 formulation as described herein is suitable for storage for at least twenty-four months.

### Example 2

### Stability of a High Concentration Liquid Formulation

In some cases, it is desirable to store an anti-IL-13 antibody formulation in a liquid format. Accordingly, the long-term stability of a liquid anti-IL-13 formulation containing a relatively high concentration of anti-IL-13 antibody was studied. Briefly, a formulation containing a humanized anti-IL-13 antibody (100 mg/ml), 10 mM histidine, 5% sucrose (weight/volume), pH 6.0 was prepared for storage by sterile filtering the formulation in depyrogenated glass vials. The formulation was stored at 2°-8° C., 15° C., or 25° C., for about six weeks, three months, six months, nine months, twelve months, eighteen months, and twenty-four months or at 40° C. for about six weeks, three months, and six months, and assayed for the presence of HMW species, LMW species, bioactivity, and concentration at each time.

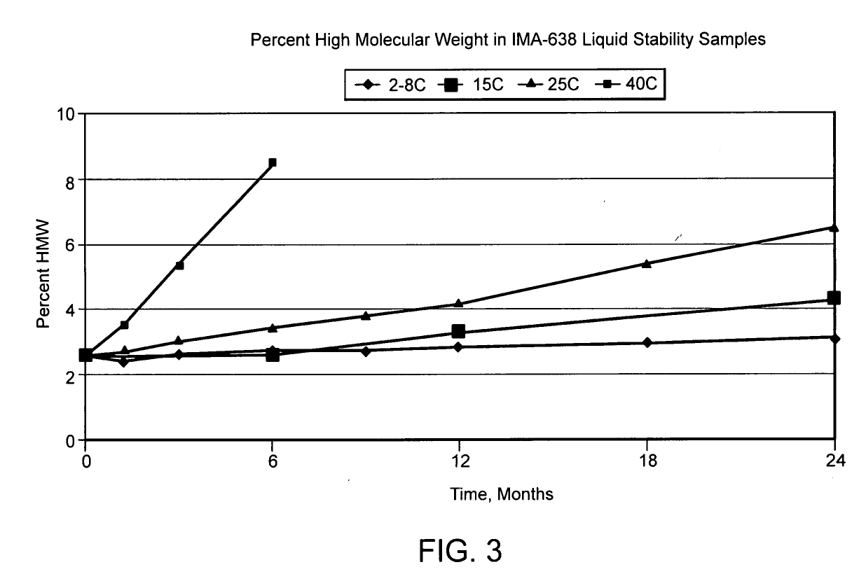

The percentage of HMW species was assayed using SEC-HPLC. The percentage of high molecular weight species in the formulation before storage was between 2%-3% of the total protein in the formulation and was between about 2%-4% in samples stored at 2°-8° C., 15° C., and 25° C. (FIG. 3) up to nine months storage, and between about 2%-4% up to twenty-four months at 2°-8° C. and 15° C. After six months of storage at 40° C., the formulation contained less than 9% HMW species (FIG. 3). Thus, there was no substantial increase in the level of HMW species in samples stored under lower temperature conditions for twenty-four months.

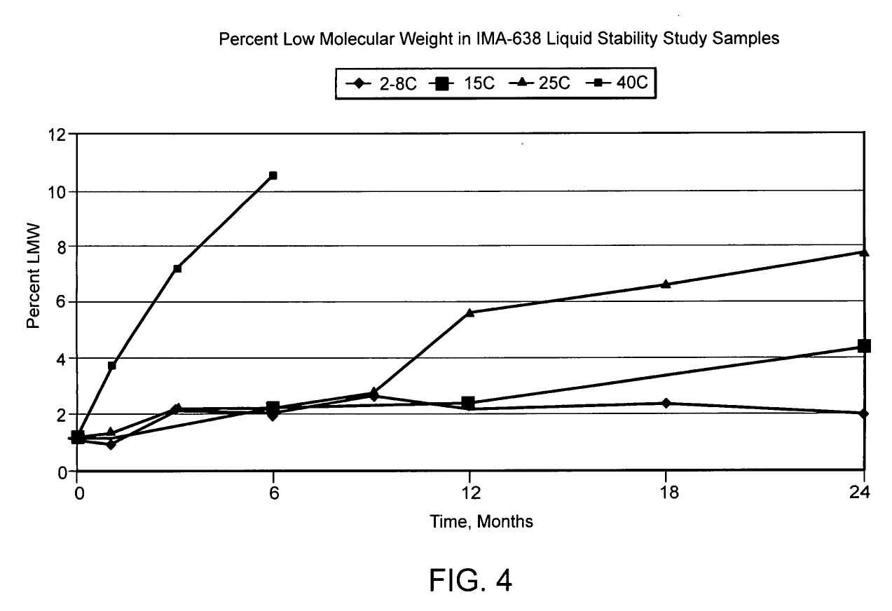

The percentage of LMW species in the anti-IL-13 antibody formulation was also assayed in the 100 mg/ml anti-IL-13 antibody formulation. The percentage of LMW species in the formulation before storage was between about 1%-2% of the total protein in the formulation prior to storage and was between about 1%-3% in samples stored at 2°-8° C., 15° C., and 25° C. (FIG. 4) up to nine months storage time, and between 1%-3% up to twenty-four months at 2°-8° C. After six months of storage at 40° C., the formulation contained less than 11% LMW species (FIG. 4). Thus, there was no substantial increase in the level of LMW species in samples stored under lower temperature conditions for twenty-four months.

Yet another stability parameter was examined using the 100 mg/ml anti-IL-13 antibody formulation: that of binding activity. In these experiments, the percentage of binding activity of the formulation was determined compared to a control after storage at 2°-8° C., 15° C., 25° C., and 40° C. for one month, three month, and six months, and nine months at 2°-8° C. and 25° C. only. The assay specifically monitors the binding affinity of the anti-IL-13 to a labeled IL-13 cytokine reagent.

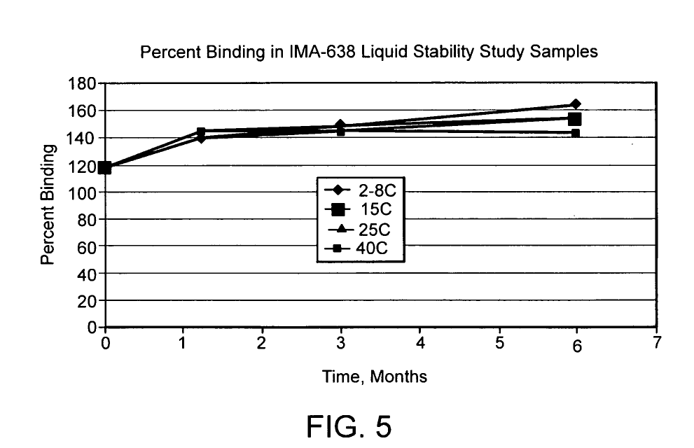

The initial binding activity of the formulation was about 120% of the reference sample and did not change substantially for any of the samples over the six-month period of testing (FIG. 5). Measured binding activity was up to about 200% of the reference, which, given the error generally observed in this assay, reflects essentially no change in the binding activity of the samples over time, and there were no temperature-related trends in binding results.

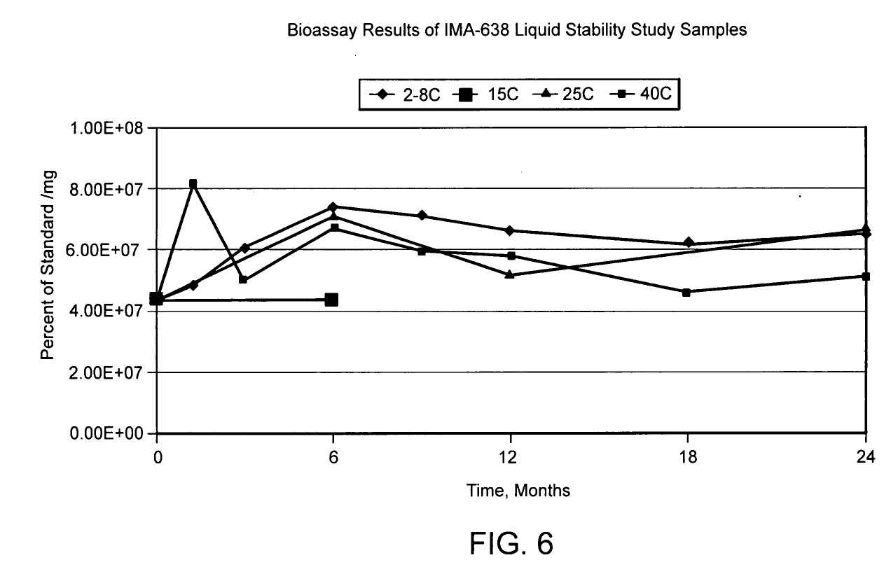

A bioassay was also used as stability parameter for the 100 mg/ml anti-IL-13 antibody formulation. The assay was conducted as described, supra, in Example 1. Samples were stored at 2°-8° C., 15° C., and 25° C. for about six weeks, three months, six months, nine months, twelve months, eighteen months, or twenty-four months or at 40° C. for about six weeks, three months, or six months. The data were expressed as binding units per milligram (FIG. 6).

Samples were about 4.5×10⁷ U/mg prior to storage and were about 4.5-7.5×10⁷ U/mg after incubation. This reflects essentially no change in the bioactivity of the samples during storage. The variability in the values reflects the variability inherent in the assay. Because there is no decrease in the amount of bioactivity in the samples, these data provide further support for the suitability of the formulation for storage of anti-IL-13.

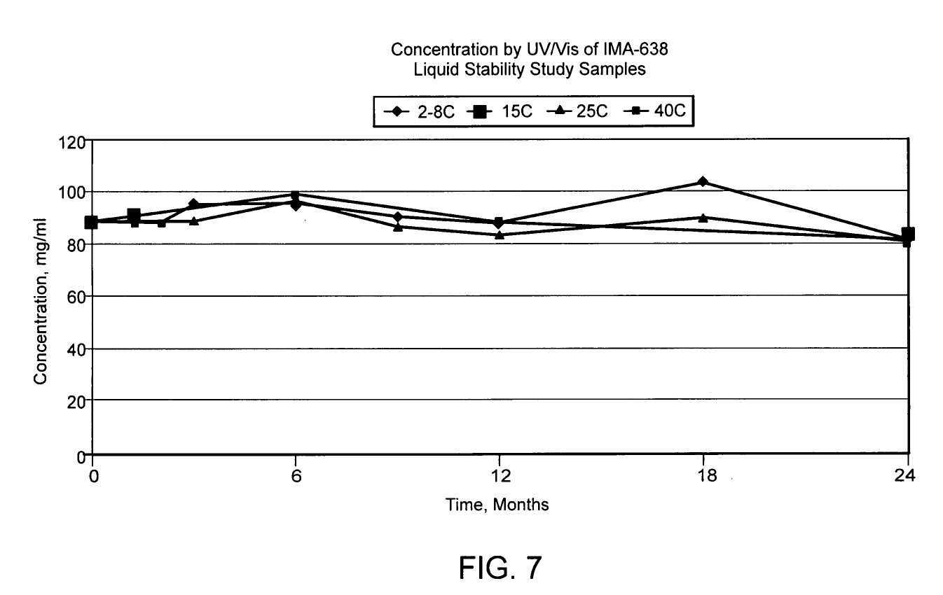

The concentration of the 100 mg/ml anti-IL-13 antibody formulation stored at 2°-8° C., 15° C., and 25° C. for about six weeks, three months, six months, nine months, twelve months, eighteen months, or twenty-four months, or at 40° C. for about six weeks, three months, or six months was also assayed by UV/Vis. The concentration of the liquid formulations at all the temperatures studied were substantially similar (FIG. 7).

### Example 3

### Storage of a Low Concentration Liquid Formulation

To further examine formulations of the invention and their suitability for storage of anti-IL-13 antibody, a formulation containing a relatively low concentration of anti-IL-13 was tested. The formulation was a liquid formulation that contained 0.5 mg/ml humanized anti-IL13 antibody, 10 mM histidine, 5% sucrose, at pH 6.0. Samples were tested after storage for six months and twelve months at 5° C., then tested for a variety of stability parameters; HMW species, LMW species, protein concentration, and binding activity. HMW species and LMW species were assayed using the methods described, supra. Protein concentration was assayed using UV-visible spectroscopy by measuring the optical density of the sample at 280 nm and subtracting scatter at 320 nm, and calculated using the molar absorptivity of the protein. The results are summarized in Table 1.

TABLE 1

| Parameter | T = 0 | Six months | Twelve months |
|---|---|---|---|
| % HMW species | 0.02% | 0.03% | 0.08% |
| (% of total) | | | |
| % LMW species | 0.12% | 0.41% | 0.79% |
| (% of total) | | | |
| Concentration | 0.44 mg/ml | 0.51 mg/ml | 0.59 mg/ml |
| % Binding activity | Not determined | 126% | 128% |
| (% of standard) | | | |

These data demonstrate that there was no substantial change in any of the assayed stability parameters, supporting the suitability of an anti-IL-13 antibody formulation containing a relatively low concentration of anti-IL-13 antibody.

### Example 4

### Suitability of an Aerosolized Anti-IL-13 Formulation

One use of an anti-IL-13 antibody formulation is for administration directly to the pulmonary system, e.g., by nebulization. To test the suitability of a formulation nebulization, a formulation of 0.5 mg/ml humanized anti-IL-13 antibody, 10 mM histidine, 5% sucrose, pH 6.0 was aerosolized using a commercially-available nebulizer, the aerosol recovered, and tested for integrity by assaying degradation (formation of HMW species), recovery using SEC-HPLC, and binding activity. The results are summarized in Table 2.

TABLE 2

| | Control (before | Post- |
|---|---|---|
| Parameter (Method) | nebulization) | nebulization |
| % HMW species | 0.75 | 0.80 |
| (SEC-HPLC) | | |
| % Recovery (SEC-HPLC) | 100% | 99% |
| Concentration | 20.7 mg/ml | 21.3 mg/ml |
| (UV-visible | | |
| spectroscopy) | | |
| % Binding activity | 189% | 186% |
| (ELISA) | | |

These data demonstrate that there was no substantial change in any of the assayed stability parameters, supporting the suitability of an anti-IL-13 antibody formulation for use as a nebulized dosage form.

### Example 5

### Mixing and Filtration

Anti-IL-13 antibody in the formulation described above was demonstrated to be robust to mixing and filtration, which are two common manufacturing unit operations. Briefly, anti-IL-13 antibody was mixed at a protein concentration of 50 mg/mL at ascending impeller speeds and times comparable to those utilized during manufacturing. Each sample collected showed no change in concentration (as assayed using UV-visible spectroscopy), high molecular weight species (assayed using SEC-HPLC) and bioactivity (assayed using a binding assay) relative to the starting material.

After the mixing study, anti-IL-13 antibody was passed through a common 0.22 μm sterilizing filter using nitrogen pressurization. In general, nitrogen pressure is below about 30 psig. After filtration, the concentration (assayed using UV-visible spectroscopy), HMW species (assayed using SEC-HPLC) and bioactivity (assayed using a binding assay) showed no change relative to the starting material.

### Example 6

### Lyophilization and Reconstitution

In one, non-limiting example of a protocol for lyophilization and reconstitution conditions for antibody, 3.2 ml of antibody at a concentration of 50 mg/ml in the formulation 10 mM histidine, 5% (50 mg/ml) sucrose, pH 6.0 is dispensed into a clear glass tubing vial (with a West 4432/50 1319 siliconized gray stopper) and freeze-dried. Upon freeze-drying, the dried contents of the vial are as follows: 160 mg antibody, 3.2×10⁻⁵ moles histidine, and 160 mg sucrose. The solid cake that results from the freeze-drying contributes approximately 0.32 ml of volume based upon the density of the solids (about 320 mg at a density of about 1 g/ml). To reconstitute the sample, 1.3 ml of water is added to the contents of the vial. The contents of the vial are solubilized in the diluent volume (1.3 ml), as well as the volume of the solids themselves (0.3 mL), for a total of about 1.6 ml, and the concentrations of the formulation is about 100 mg/ml antibody, about 20 mM histidine, and about 10% sucrose, pH 6.0.

### Example 7

### Preparation and Lyophilization of Samples

Anti-IL-13 antibody Sample Preparation

A frozen sample of a humanized anti-IL-13 antibody at a concentration of about 85 mg/mL in 20 mM histidine, 10% sucrose pH 6.0 was thawed in a 37° C. water bath. A 125 mL aliquot of the thawed material was dialyzed against 10 mM histidine, 5% sucrose, pH 6.0 using 6 kD-8 kD molecular weight cut-off Spectra/Por dialysis tubing. The resulting solution was diluted to a target of 50 mg/mL with 10 mM histidine, 5% sucrose, pH 6.0 (the anti-IL-13 antibody formulation for use as a drug).

Lyophilization Practices

In all runs, an aluminum foil shield in front of the door and a shelf height of 63 mm was used to minimize radiation within the lyophilizer. In all runs, one tray was entirely filled to maintain a consistent load on the lyophilizer. Stoppers were autoclaved and dried for all protein vials. All vials for protein samples were rinsed with de-ionized water and depyrogenated. Vials and stoppers that were used to fill the remainder of the tray were untreated.

Vials seeded with the anti-IL-13 antibody formulation were prepared aseptically in a biosafety cabinet at a target of 160 mg/vial. Vials for stability studies were filled with 3.2 ml of fresh formulation described in Example 6 prior to each run (material that had not been previously lyophilized). During lyophilization, additional vials were filled with suitable buffers that were compatible with the target lyophilization cycle to maintain a consistent load on the lyophilizer. Lyophilization was monitored through the use of thermocouples within the protein array.

Modulated Differential Scanning Calorimetry (mDSC)

All samples for mDSC were run in modulated mode with an amplitude of 0.5° C. and a period of 100 seconds. For post-lyophilization powders, samples were heated at 2° C./min. to 150° C. All powder samples were prepared using a nitrogen-purged glove box. For liquid samples, all temperature ramps were performed at 0.5° C./min. and temperatures were matched to those utilized in the lyophilization cycles. The final heating ramp was performed at 2° C./min. to magnify the glass transition. Liquid samples were prepared on the laboratory bench.

Freeze-Drying Microscopy

To perform freeze-drying microscopy, a sample was frozen to −40° C. at 0.5° C./min., to mimic lyophilization. After vacuum initiation, the temperature was gradually increased to observe structural changes in the sample as a function of temperature during sublimation. The freeze-drying microscope does not allow for pressure control, so the sample was dried under complete vacuum.

Moisture Analysis

Karl Fischer titration was used to assay moisture in lyophilized samples. Lyophilized samples were reconstituted with 3 ml methanol. Duplicate or triplicate injections of 500 μL were performed. A 1% water standard was injected post use as a suitability check.

Fourier Transform Infrared Spectroscopy (FTIR)

FTIR measured secondary structure of the antibody in the dry powder state. A pellet containing approximately 1 mg of formulated, dried protein dispersed within 300 mg KBr was pressed and scanned 200 times. After data collection, analysis involved spectral subtraction of sucrose placebo, baseline correction, smoothing, second derivative, and area normalization.

Stability

The stability of lyophilized antibody in formulations was assessed as a function of storage time and temperature. Samples of lyophilized anti-IL-13 antibody were assayed post-lyophilization, after four weeks of storage at 2° C.-8° C. and after two weeks and four weeks of storage at 50° C. Refrigerated samples were stored in a walk-in refrigerated cold room. High temperature samples were stored in a Lab Line Imperial Incubator set at 50° C. At the appropriate time points samples were removed from storage and allowed to warm up or cool down to room temperature before assaying.

Reconstitution and Visual Appearance

Vials of lyophilized formulations from both post-lyophilization analysis and storage stability analysis were visually inspected before, during, and after being reconstituted with 1.2 ml of sterile water for injection. Vials were inspected in a light box against both a black and a white background for cake color, integrity, moisture, particulates, and defects before reconstituting. After visually inspecting the lyophilized cake, the cap and crimp seal were removed from the vial using a de-crimper. The stopper was removed and the sterile water for injection was slowly dispensed into the vial using an appropriate pipette. The diluent was dispensed using a swirling motion to ensure full wetting of the cake. Once the diluent was completely dispensed, timing of reconstitution was initiated with a standard laboratory timer and the vial was restoppered. Reconstitution was complete when the final piece of solid dissolved. Rolling the vial between one's hands facilitated reconstitution. As the lyophilized cake was in the process of reconstituting, observations about the state of the dissolving solution such as clarity, bubbling, and foaming were recorded. Once reconstitution was complete, the reconstitution time was recorded and the vials were left on the bench for several minutes so that the resulting solution could settle and the majority of bubbles formed during reconstitution could dissipate. The reconstituted solution was then inspected in a light box against both a black and a white background for color, clarity, and particulates.

High Performance Size Exclusion Chromatography (SEC-HPLC)

Two microliters of neat samples of anti-IL-13 antibody formulation were injected onto a G3000sw×1 column with a guard column (TosoHaas Part Nos. 08541 and 08543). The mobile phase was phosphate buffered saline (PBS) with 250 mM sodium chloride added. The flow rate was 0.75 ml/min. and the run time was 30 minutes. The ultraviolet absorbance was monitored at a wavelength of 280 nm. The chromatogram was integrated to separate the main anti-IL-13 antibody peak from high and low molecular weight species using Waters Empower™ software.

Ultraviolet-Visible Absorbance Spectroscopy for Concentration Determination (A₂₈₀)

Samples of the formulation having antibody at a concentration of 100 mg/ml were diluted to approximately 0.5 mg/ml and 0.25 mg/ml by adding 10 μl of sample to 1990 μl and 3990 μl of 10 mM histidine, 5% sucrose, pH 6.0, respectively. Two hundred microliters of the resulting solutions were placed in individual wells in a 96-well microplate along with a buffer blank. The plate was read in a Spectramax® Plus plate reader for ultraviolet absorbance at wavelengths of 280 nm and 320 nm. Subtracting the 320 nm absorbance from the 280 nm absorbance and dividing by the extinction coefficient (1.405 mL/mg-cm) multiplied by the path length (1 cm) determined protein concentrations of the solution in each well. The appropriate dilution factor was applied, and an average protein concentration was determined.

Ultraviolet-Visible Absorbance Spectroscopy for Light Scatter (A₄₂₀)

Two hundred microliters of each anti-IL-13 antibody sample to be analyzed was aliquoted into individual wells on a 96-well microplate. A buffer blank served as a control. The plate was read in a Spectramax Plus plate reader for visible absorbance at a wavelength of 420 nm.

Electrochemiluminescence (ECL) Binding Assay Samples were subjected to binding analysis utilizing the E. coli Flag anti-IL-13 antibody binding assay format (BioVeris, Gaithersburg, Md.). The assay was performed on samples aliquoted into a 96-well plate format.

Anti-IL-13 Antibody Bioassay

Samples were tested for bioactivity using a TF-1 cell proliferation bioassay. The IL-13 antibody blocks binding of IL-13 cytokine to cell surface receptors in vivo preventing the activation of receptor bearing cells involved in pathogenesis of allergic diseases and asthma. The in vitro bioassay model used in this study consists of a cell line (human TF1 erythroleukemia cell line; ATCC CRL-2003) that expresses IL-13 receptor and proliferates in the presence of IL-13 cytokine.

Inhibition of the IL-13 response of TF1 cells by the IL-13 antibody was fitted using a 4-parametric logistic equation. The biological activity (relative potency) is determined by comparing the inhibition curve of the IL-13 antibody test sample to the inhibition curve of reference material used as an assay standard.

Cycle Development Strategy

A series of sequential steps (described below) were used to develop a lyophilization cycle.

Critical Product Temperature Identification

The critical product temperature for an anti-IL-13 antibody was identified by two orthogonal methods—modulated Differential Scanning Calorimetry (mDSC) and Freeze-Drying Microscopy. These two methods are used to identify the glass transition temperature of the frozen product (mDSC) and the resulting collapse (Freeze-Drying Microscopy). A lyophilization cycle that maintains the product below this temperature during primary drying should yield an intact cake structure. The lowest temperature suitable temperature was assumed to be −25° C., and so this temperature is generally included in procedures designed to test conditions and formulations when developing a formulation and methods for lyophilization of an antibody as described herein.

### Lyophilization Cycle Execution

Based on the results from the studies described, supra, three different lyophilization cycles were performed to examine three parameters of interest in developing a suitable lyophilization procedure for preparing a lyophilized formulation suitable for storage or other procedures. The first parameter examined was control cycle, which repeats cycles from previous stability studies. All prior developmental stability cycles utilized this cycle, so it served as a starting point for this analysis.

The second parameter tested was the impact of annealing. The reconstitution time for anti-IL-13 antibody formulation lyophilized using the control cycle above is fairly long, e.g., about 100 sec. to 500 sec (FIG. 16). Inclusion of annealing above the glass transition temperature of the frozen solution as an additional step during the frozen thermal treatment serves to increase the ice crystal size prior to vacuum initiation. This increased ice crystal size leads to an increased pore size of the dried cake at the conclusion of lyophilization. Larger pores can allow for improved water penetration into the lyophilized cake and improve reconstitution.

The third tested parameter was an aggressive cycle. Increasing the primary drying temperature significantly above the control cycle set point can significantly increase the anti-IL-13 antibody formulation product temperature during primary drying. This lyophilization cycle serves as an evaluation of the sensitivity of an anti-IL-13 antibody formulation to product temperature during lyophilization, and can be used in evaluation of manufacturing deviations during early clinical lots prior to the execution of formal lyophilization robustness studies.

Assessment of Lyophilization Cycles

The assessment of the selected lyophilization cycles on anti-IL-13 antibody formulations was split into two aspects: immediate comparison based on tests performed post-lyophilization, and potential longer-term impact caused after incubation under accelerated conditions.

Critical Product Temperature Identification

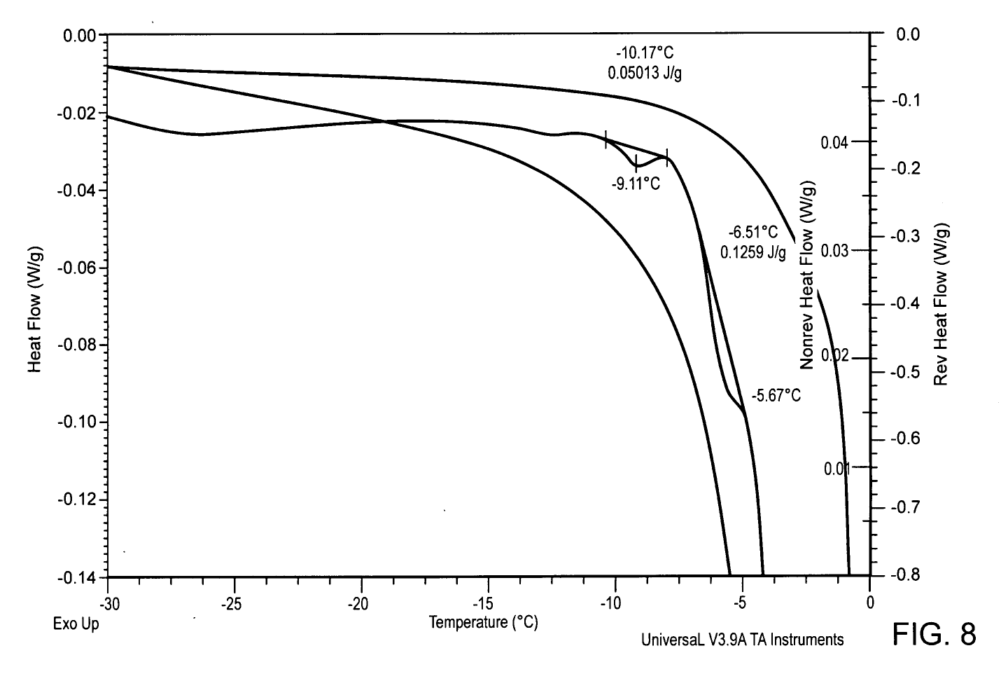

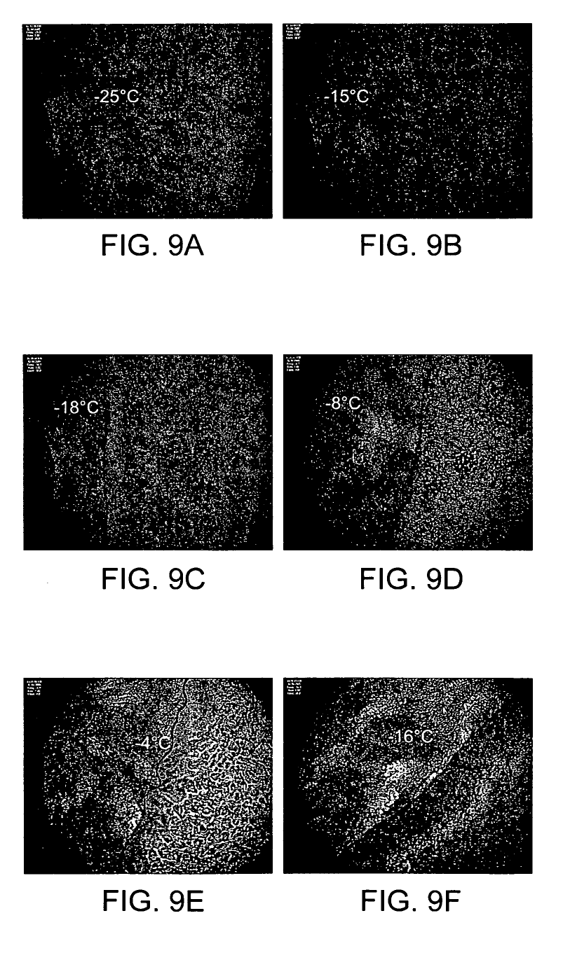

The anti-IL-13 antibody formulation product contained nearly 50% protein. As such, the protein was anticipated to dominate the physical properties of the frozen and lyophilized states. Prior to lyophilization, sub-ambient modulated Differential Scanning Calorimetry (mDSC) searched for the glass transition temperature of the freeze-concentrated amorphous phase of the formulation. In this experiment, anti-IL-13 antibody was at a concentration of 50 mg/ml in 5% sucrose, 10 mM histidine, pH 6.0. Under these conditions, the lowest identified transition was at −11° C. (FIG. 8). The critical temperature was confirmed by assaying the freeze-drying microscopy temperature progression (FIGS. 9A-9F). In these experiments, structure was lost by heating from −25° C. to −15° C. and was regained by cooling to −18° C. Structure was further lost by heating from −10° C. to the onset of melting at −4° C. All of the changes were reversible, as indicated by the comparable structure observed upon cooling the sample to −16° C. Thus, a reversible transition was observed at about −15° C., and another transition between −10° C. and −6° C. Reducing the temperature below −16° C. leads to a dried structure comparable to the original structure. Based on this information, a product temperature of −15° C. was selected as the critical temperature to remain below during lyophilization. This method illustrates a method for selecting a critical temperature for lyophilization.

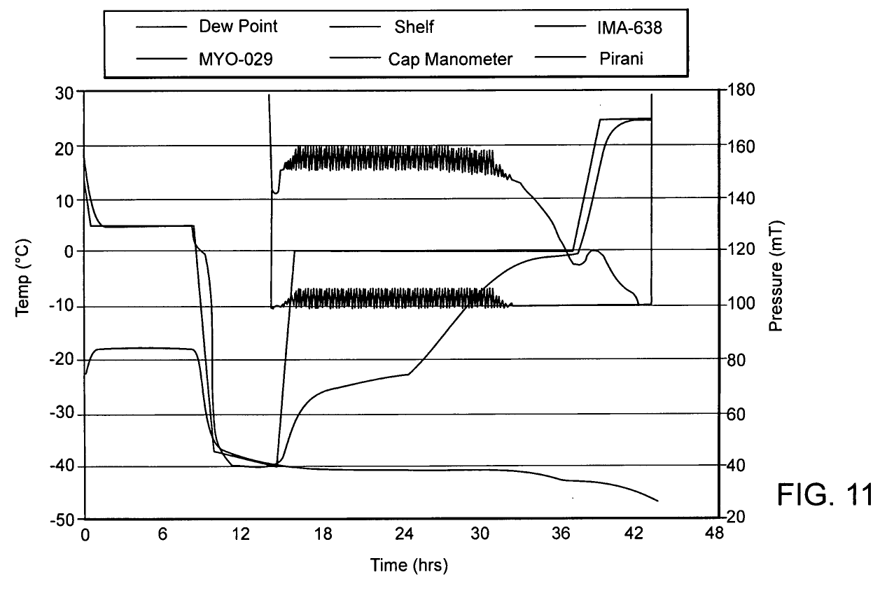

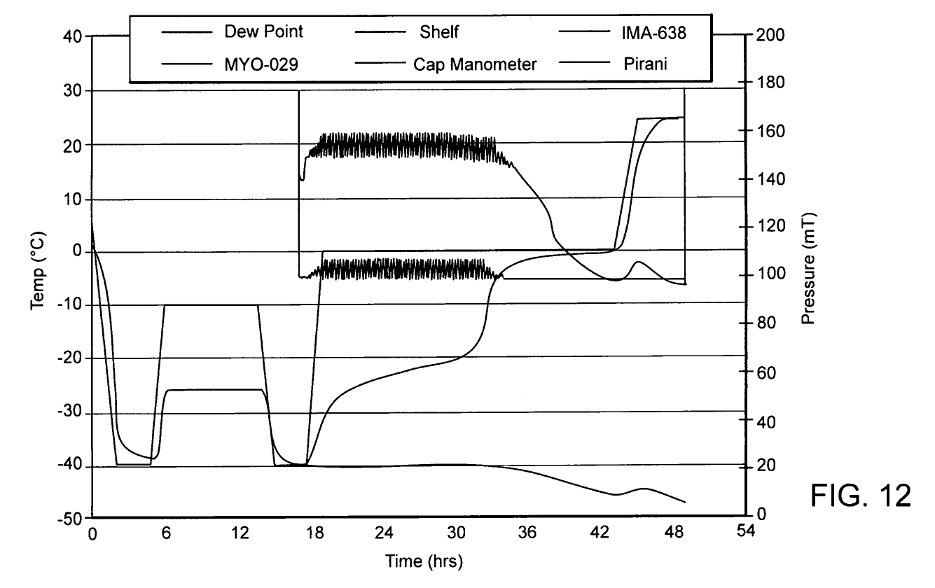

Three lyophilization cycles were executed consecutively. The cycle traces are shown in FIGS. 10-12. All cycles maintained a chamber pressure of 100 mT during primary and secondary drying. Ramp rates were 0.5° C./min. for all ramps except between primary and secondary drying in FIGS. 11 and 12, which was 0.2° C./min. for those cycles). The varied parameters are summarized in Table 3.

TABLE 3
Comparison of lyophilization parameters (Primary drying time for
last thermocouple to reach shelf temperature)

| Step | Aggressive | Control | Anneal |
|---|---|---|---|
| Annealing | — | — | 8 hrs |
| 1° Drying | 12 hrs | 21 hrs | 21 hrs |
| 2° Drying | 3 hrs | 4 hrs | 4 hrs |

Assessment of Lyophilization Cycles: Post Lyophilization

The product (anti-IL-13 antibody) temperature profile during primary drying for each of the three cycles (control, aggressive, and annealing) is shown in FIG. 13. The annealing and control product thermocouples were similar, while the elevated shelf temperature of the aggressive cycle led to an increase of nearly 10° C. during primary drying.

After lyophilization, vials of anti-IL-13 antibody formulation from each of the three lyophilization cycles were tested for biochemical integrity, both as a solid and as a reconstituted liquid. The solid state was assessed using the following methods; mDSC (measure glass transition temperature), BET surface area measurement, Karl Fischer moisture titration, Fourier-Transfer Infrared Spectroscopy (measure protein secondary structure), and cake appearance. Reconstituted liquids were assessed by reconstitution time, visual appearance, ultraviolet absorbance at 280 nm for protein concentration, visible absorbance at 420 nm for light scatter, SEC-HPLC for high molecular weight quantitation, CEX-HPLC for surface charge heterogeneity and IGEN binding, and TF-1 bioassay for biological activity.

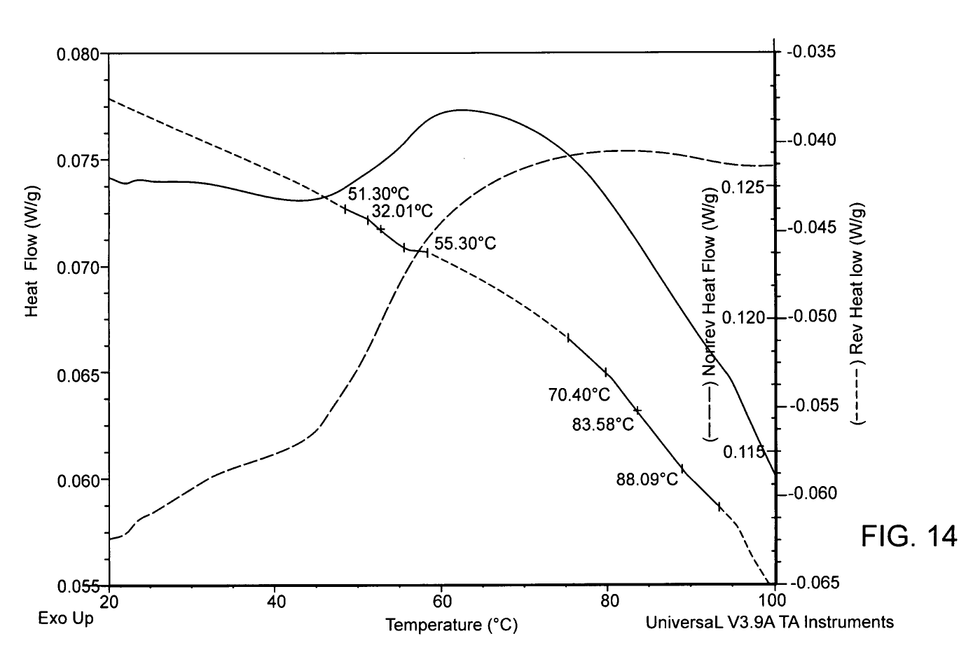

All three cycles produced white solid cakes with no apparent defects including particulates or moisture. The mDSC thermogram for the control cycle is shown in FIG. 14. Table 5 summarizes the results for each cycle for the primary thermal transition. The transition at 53° C. was not as large in magnitude, but still detectable, in the other two lyophilization cycles. This transition did not appear to impact the stability of the protein upon accelerated storage at 50° C.

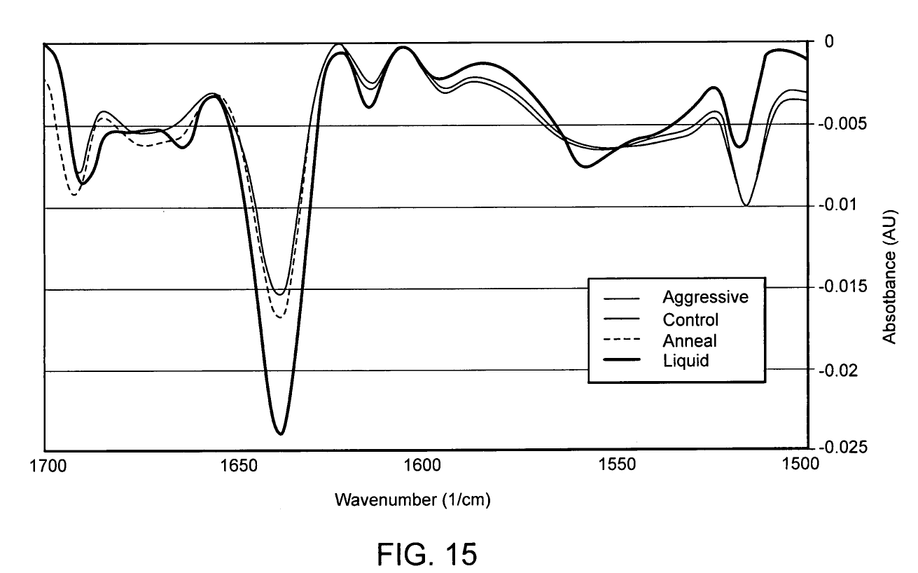

Comparing the secondary structure of the formulations post-lyophilization revealed that the protein secondary structure is comparable between the three samples (Table 4, FIG. 15). In FIG. 15, which shows the second derivative of powder Fourier transform infrared spectroscopy (FTIR) in the amide I region of the sample antibody, the cumulative area of each scan was normalized to 1. The information included in Table 4 represents the fraction of the total area in the β-sheet band (1624-1657 cm⁻¹) as a basis for comparison between samples. When comparing the secondary structure in the dried state against the formulation in the liquid state, the difference in relative β-sheet area was noticeable (0.37 as a liquid vs. 0.25-0.27 as a lyophilized powder). This difference is most likely due to the absence of water in the lyophilized state and the corresponding change in protein conformation.

TABLE 4
Measured Glass Transition Temperature (Tg), BET
surface area, residual moisture and secondary structure post
lyophilization

| | | | | Depth of |
|---|---|---|---|---|
| | | BET Surface | | β-sheet |
| Cycle | T_{g} (° C.) | Area (m²/g) | Moisture | band |
| Aggressive | 86 | 0.48 | 0.45% | 0.255 |
| Control | 84 | 0.64 | 0.73% | 0.249 |
| Anneal | 85 | 0.59 | 0.59% | 0.270 |

One vial from each cycle was reconstituted with 1.2 ml of sterile water for injection. Appearance during reconstitution, reconstitution time, and appearance 60 mins. post reconstitution were recorded for each cycle and are summarized in Table 6. All three cycles required physical agitation (rolling between hands) to solubilize the cake. The cakes for the aggressive cycle (cycle 1) and the control cycle (cycle 2) began to break up and dissolved within a timeframe useful for production: reconstitution time was 140 sec. and 73 sec., respectively. Much of the reconstitution time was spent dissolving smaller, more stubborn pieces of cake. The annealing cycle sample (cycle 3) took the longest time to reconstitute. The result refutes the theory that an annealing step would result in a shorter reconstitution time presumably due to the formation of a more porous cake. The cake remained intact upon reconstitution and slowly dissolved in 373 sec., resembling a dissolving Lifesaver™. All three cycles produced varying amounts of foam during reconstitution. The control cycle produced the most amount of foaming followed by the annealing cycle then the aggressive cycle as seen by solution scatter by UV/Vis at 420 nm (see Table 5). Once reconstituted, the samples were allowed to settle for 60 minutes. By that time, much of the foam had dissipated and all three solutions had a similar appearance when inspected using a light box against both a black and a white background. All three cycles had a yellow tint and were slightly opalescent with the annealing sample being somewhat more opalescent.

All three samples were assayed for biochemical integrity using assays described herein. These data demonstrated that there are no apparent differences in the integrity of an anti-IL-13 antibody formulation, post reconstitution, as a function of the lyophilization cycle. The amount of protein recovered as demonstrated by measuring the concentration of antibody in the formulations was essentially equal for all three cycles. The amount of high molecular weight compounds in a formulation as measured by size exclusion chromatography and the amount of surface charge heterogeneity as measured by cation exchange chromatography was essentially the same for all three cycles. There were no identified changes in the functionality of the molecule as measured by the IGEN binding assay and the TF-1 bioassay as a function of lyophilization cycle.

TABLE 5
Post-Reconstitution Data

| | 1.2-mL reconstitution (100 mg/mL target accounting for cake) | | |
|---|---|---|---|
| | Cycle 1 | Cycle 2 | Cycle 3 |
| Assay | (aggressive) | (control) | (control w/8 hr anneal) |
| Appearance | Quite foamy dissipating | Extremely foamy dissipating | Extremely foamy, larger |
| during | quickly. Chunks of cake | less quickly. Chunks of cake | bubbles, dissipating much |
| recon | difficult to recon. Shaken | difficult to recon. Shaken | slower. Cake maintains shape |
| | vigorously to get into solution | vigorously to get into solution | during very slow recon. |
| | | | Shaken vigorously to get into |
| | | | solution |
| Appearance | Slightly opalescent with | Visibly more opalescent with | Slightly opalescent with |
| after recon | yellow tint. Bubbles still | yellow tint. Bubbles still | yellow tint. Bubbles still |
| (60 minutes) | remain. | remain | remain |
| Recon Time | 140 seconds | 73 seconds | 373 seconds |
| A420 | 0.227 | 0.518 | 0.257 |
| A280 | 103.6 | 100.5 | 104.1 |
| (mg/mL) | | | |
| SEC-HPLC | 1.1 | 1.1 | 1.1 |
| % HMW | | | |
| IGEN | 153 | 153 | 164 |
| % Binding | | | |
| Specific | 6.0E+07 | 5.8E+07 | 6.8E+07 |
| Activity | | | |
| (U/mg) | | | |

Stability

Although there did not appear to be an immediate post-lyophilization impact on the integrity of an anti-IL-13 antibody in the formulation described herein as a function of the lyophilization cycles investigated, it is important to assess whether storage stability varies as a function of the lyophilization cycle. To test this, a short-term accelerated stability study was executed as outlined in the section above “Stability.” Samples were monitored for reconstitution time, changes in protein concentration by UV/Vis at 280 nm, changes in solution light scattering by UV/Vis at 420 nm, changes in high molecular weight aggregate by SEC-HPLC, and changes in binding activity by IGEN binding assay.

FIG. 16 is plotted to show reconstitution time as a function of storage time and storage temperature. Although there is variability in the absolute numbers for reconstitution time, the trend, with the exception of the aggressive cycle and the annealing cycle stored at 5° C., is similar to what was observed in the post-lyophilization analysis. The control cycle samples reconstituted most quickly, followed by the aggressive cycle samples. The annealing cycle samples were the slowest to reconstitute. The variability from time point to time point and the deviation from the post-lyophilization trend by the aggressive and annealed samples stored at 5° C. could be due to one or more poorly-controlled variables. These include the rate at which the cake is wetted during reconstitution, how much and what part of the cake is wetted as the water for injection is dispensed into the vial, and how aggressively the vial is agitated during reconstitution. All of these variables are subjective and operator-dependent, and may have impacted reconstitution time and light scattering.

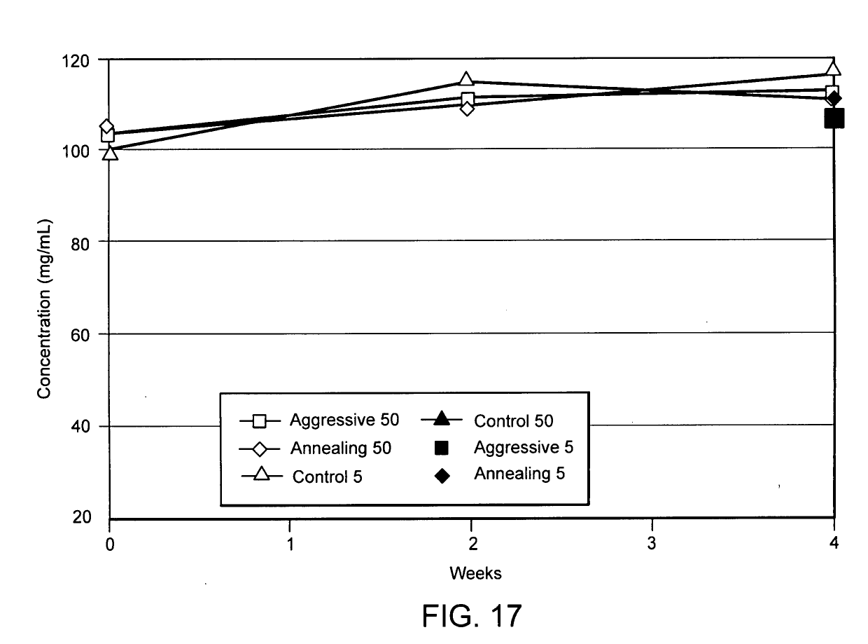

Protein concentration, shown in FIG. 17, did not vary significantly between the three cycles tested over the course of storage (zero to four weeks) or as a function of temperature (5° C. and 50° C.). The increase in concentration from the initial time point to two weeks may have been due to differences in the accuracy of the measurement of the reconstitution volume from one time point to the next.

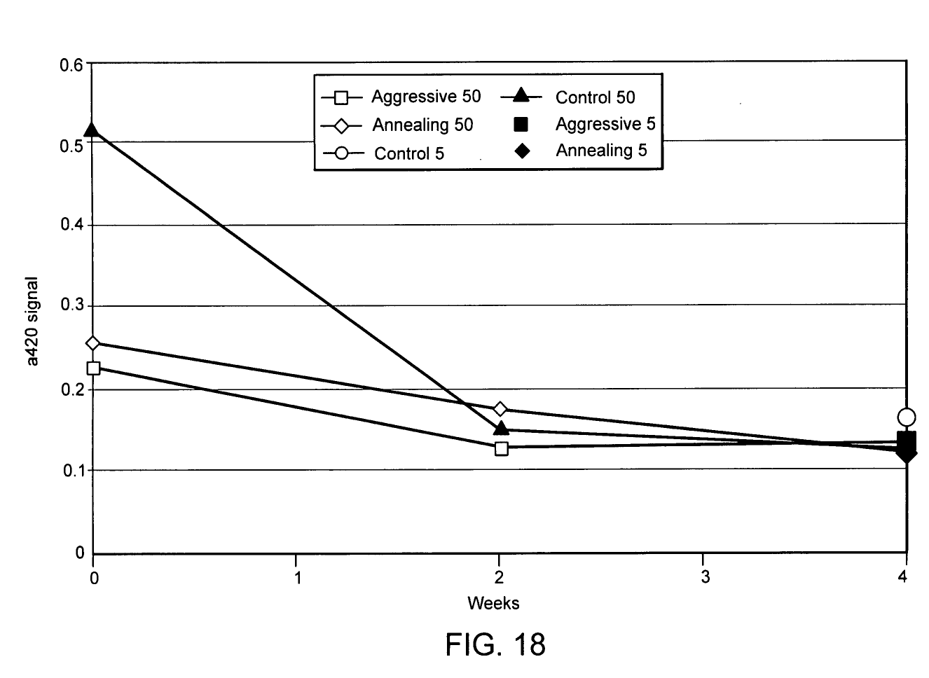

Solution scatter, shown in FIG. 18, did not vary significantly between the three cycles over the course of storage or as a function of temperature. The elevated result at the initial time point for the control cycle was due to additional bubble entrainment due to sample handling, rather than a result of cycle differences.

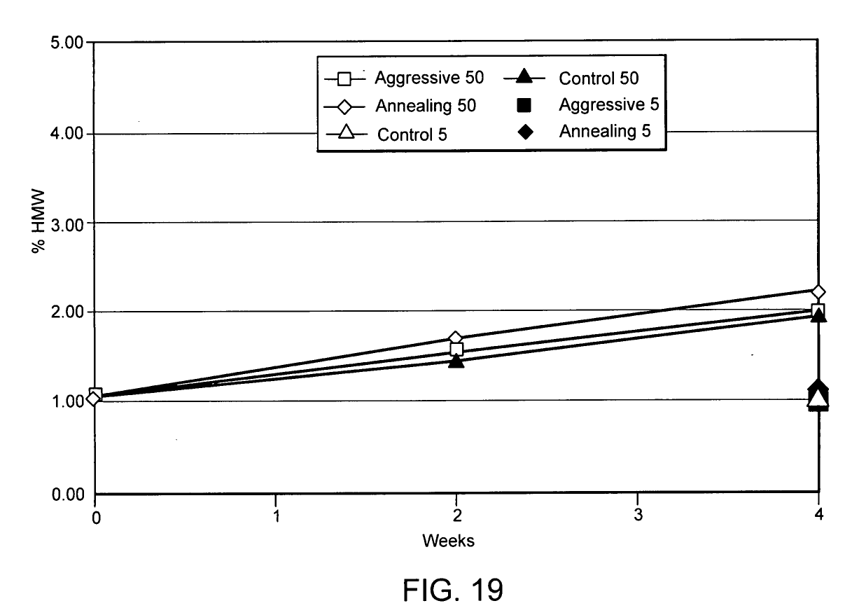

Samples were also assayed for the percentage of HMW species present during storage. The assays were performed using SEC-HPLC. The data, as shown in FIG. 19, demonstrate that the percentage of high molecular weight aggregates did not vary significantly during storage between the three different lyophilization cycles.

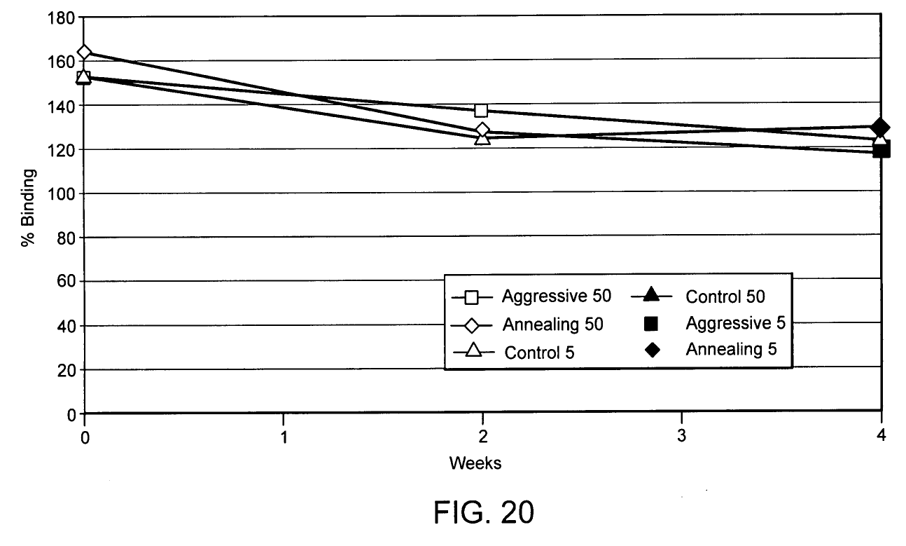

The samples were also assayed for binding using a plate assay in a 96-well format (IGEN). FIG. 20 shows that the binding of the anti-IL-13 antibody in the formulation did not change significantly as a function of lyophilization cycle over the course of four weeks at either 2°-8° C. or 50° C.

These data demonstrate that the anti-IL-13 antibody in the formulation has a comparable stability profile as a function of the three lyophilization cycles investigated. The addition of an annealing step appears to worsen reconstitution rather than improve it. The aggressive cycle will act as a robustness assessment due to the observed increase in product temperature of nearly 10° C. during primary drying.

### CONCLUSION

The anti-IL-13 antibody in the formulation was demonstrated to be robust during lyophilization to extremes in product temperature. The stability profile upon storage at 50° C. for four weeks was about identical for material that had nearly 10° C. differences in product temperature during primary drying.

### Example 8

### IL-13 Antibody Formulation

In order to screen for possible excipients for an IL-13 antibody liquid formulation, a short term accelerated stability study was conducted using 0.5 ml of a 100 mg/ml IMA-638 antibody in either 13 mm West glass vials with West 4432/50 stoppers or BD Hypak™ pre-fillable syringes at a storage temperature of 40° C. for six weeks. The stability of the antibody was then tested by measuring the concentration using the absorbance at 280 nm and by SEC-HPLC.

The formulations tested included varying the pH from 5.0 to 5.5 to 6.0; different buffers such as histidine, sodium succinate, and sodium acetate; different sucrose concentrations (0%, 2.5%, 5.0%, and 10%); and other additives such as sorbitol, glycine, arginine, and methionine. Table 6 below provides the formulations that were tested in this screen.

TABLE 6
Liquid Formulations

| No. | Formulation |
|---|---|
| 1. | 10 mM Histidine, 0% Sucrose, pH 6.0 |
| 2. | 10 mM Histidine, 2.5% Sucrose, pH 6.0 |
| 3. | 10 mM Histidine, 5% Sucrose, pH 6.0 |
| 4. | 10 mM Histidine, 10% Sucrose, pH 6.0 |
| 5. | 10 mM Histidine, 0% Sucrose, pH 5.5 |
| 6. | 10 mM Histidine, 2.5% Sucrose, pH 5.5 |
| 7. | 10 mM Histidine, 5% Sucrose, pH 5.5 |
| 8. | 10 mM Histidine, 10% Sucrose, pH 5.5 |
| 9. | 10 mM Histidine, 5% Sorbitol, pH 6.0 |
| 10. | 10 mM Histidine, 1% Glycine, pH 6.0 |
| 11. | 10 mM Succinate, 5% Sucrose, pH 6.0 |
| 12. | 10 mM Acetate, 5% Sucrose, pH 5.0 |
| 13. | 10 mM Acetate, 5% Sucrose, pH 5.5 |
| 14. | 10 mM Histidine, 5% Sucrose, 2% Arginine, pH 6.0 |
| 15. | 10 mM Histidine, 5% Sucrose, 100 mM Methionine, pH 6.0 |

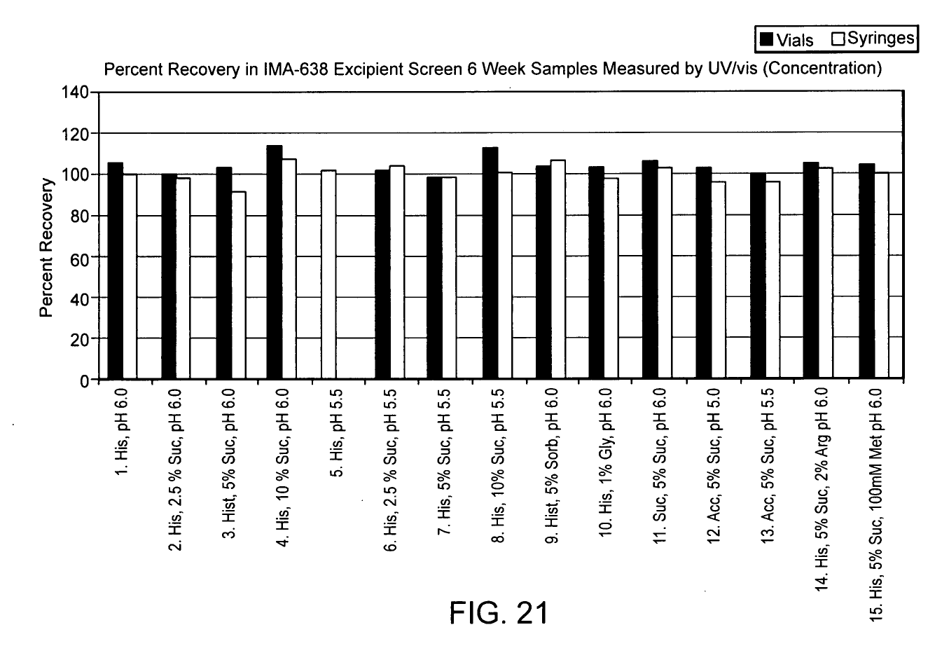

The percent recovery over six weeks of storage at 40° C. was assessed by determining concentration of the antibody by UV/Vis and is shown in FIG. 21. The recovery was substantially similar amongst the formulations but the highest recovery was obtained in Formulations 4 and 8.

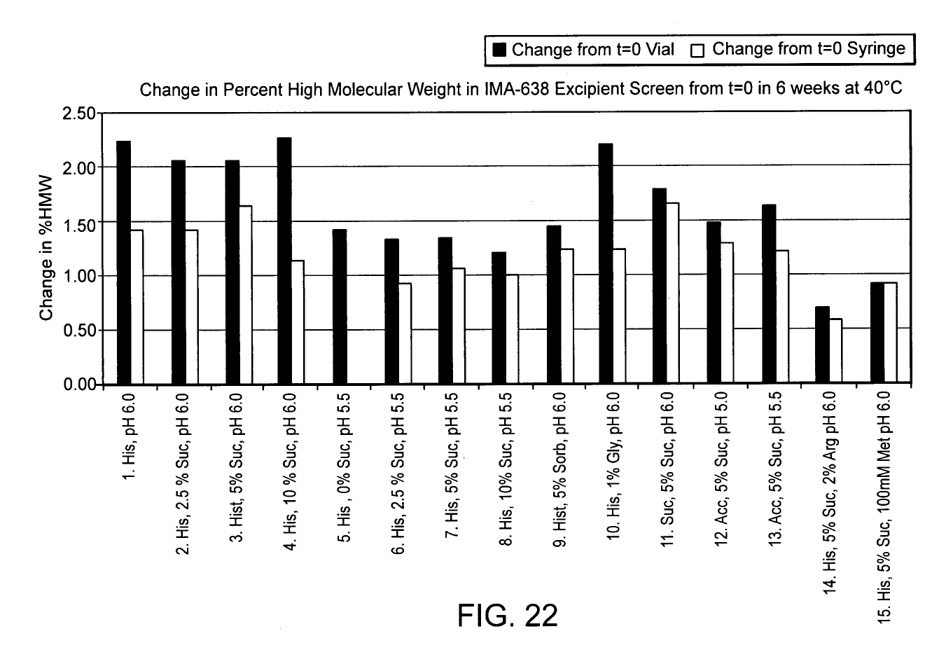

The percent increase in high molecular weight species over six weeks of storage at 40° C. is shown in FIG. 22. The pre-filled syringes had fewer high molecular weight aggregates compared to the vials (see, FIG. 22, Formulation 4). Formulations 6, 8, 14, and 15 showed the smallest increase in high molecular weight species (between 0.5% and 1.25%).

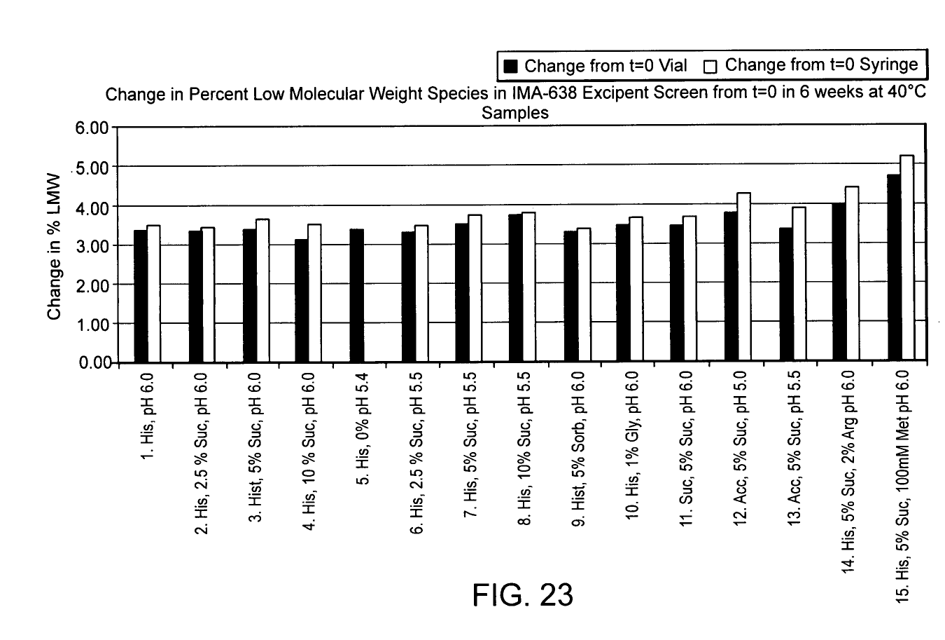

The percent increase in low molecular weight species over six weeks of storage at 40° C. is shown in FIG. 23. In contrast to the HMW, the pre-filled syringes generally had a small increase in LMW species compared to the glass vials. Formulations 1-13 had a change in % LMW of about 3%-4%.

In conclusion, most of the formulations demonstrated acceptable stability profiles, confirming an optimal pH of 5-6.5, and allowing for inclusion of different suitable excipients—as none of the excipients were detrimental to the stability of the protein.

### Example 9

### Assessment of the Need for Tween in the Formulations

To address whether Tween is needed in the lead candidate formulations from Example 8 in the context of interfacial degradation, a shaking study and a freeze-thaw study were conducted using the eight lead candidates which are listed in Table 7.

TABLE 7
Lead Candidates

| No. | Formulation |
|---|---|
| 1. | 10 mM Histidine, 0% Sucrose, pH 6.0 |
| 2. | 10 mM Histidine, 5% Sucrose, pH 6.0 |
| 3. | 10 mM Histidine, 10% Sucrose, pH 6.0 |
| 4. | 10 mM Histidine, 5% Sucrose, 0.01% Tween 80, pH 6.0 |
| 5. | 10 mM Histidine, 5% Sucrose, 2% Arginine, pH 6.0 |
| 6. | 10 mM Histidine, 5% Sucrose, 2% Arginine, 0.01% Tween 80, |
| | pH 6.0 |
| 7. | 10 mM Histidine, 5% Sucrose, 70 mM Methionine, pH 6.0 |
| 8. | 10 mM Histidine, 5% Sucrose, 70 mM Methionine, 0.01% |
| | Tween 80, pH 6.0 |

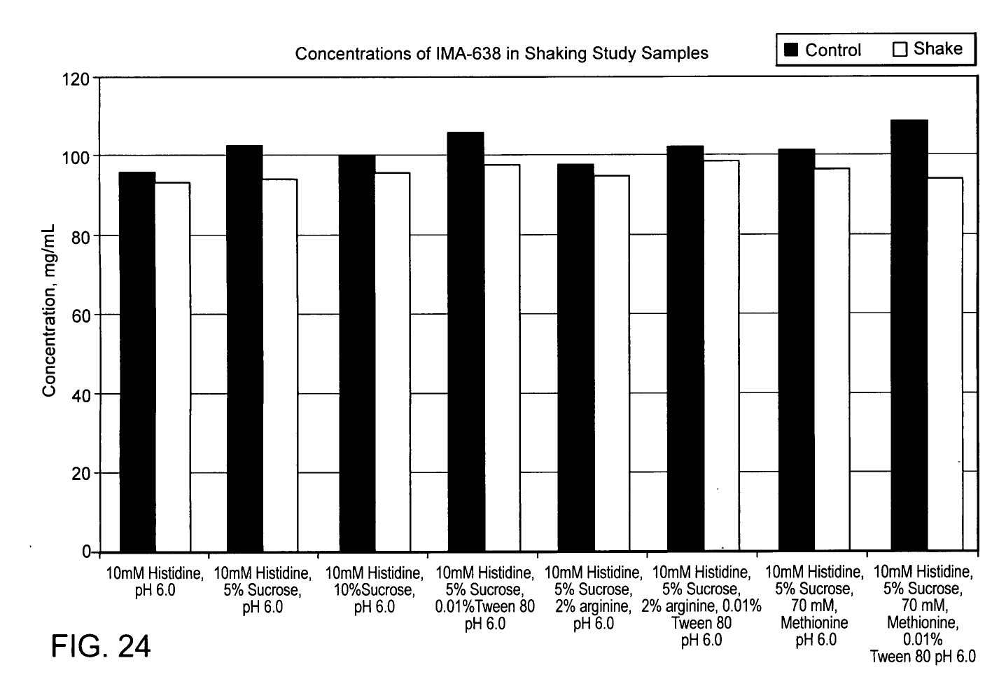

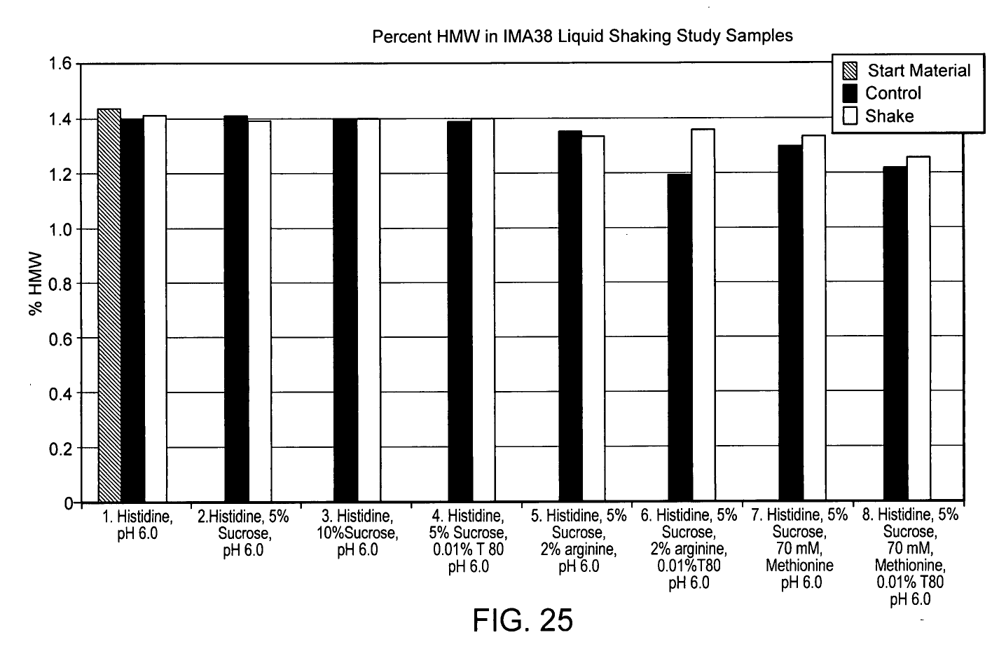

The shaking study was conducted by using 0.25 ml of 100 mg/ml IMA-638 liquid formulations in glass vials and shaking the glass vials at room temperature on a gel shaker at approximately 200 rpm for twenty-four hours. The concentration of the samples that were shaken were compared with samples that were not shaken (Control). The concentration of IMA-638 following shaking of the different antibody formulations is shown in FIG. 24. The concentrations were substantially similar amongst the formulations. FIG. 25 provides the % HMW species following shaking of the IMA-638 formulations. The HMW species amongst the formulations ranged between about 1.2% to about 1.5%.

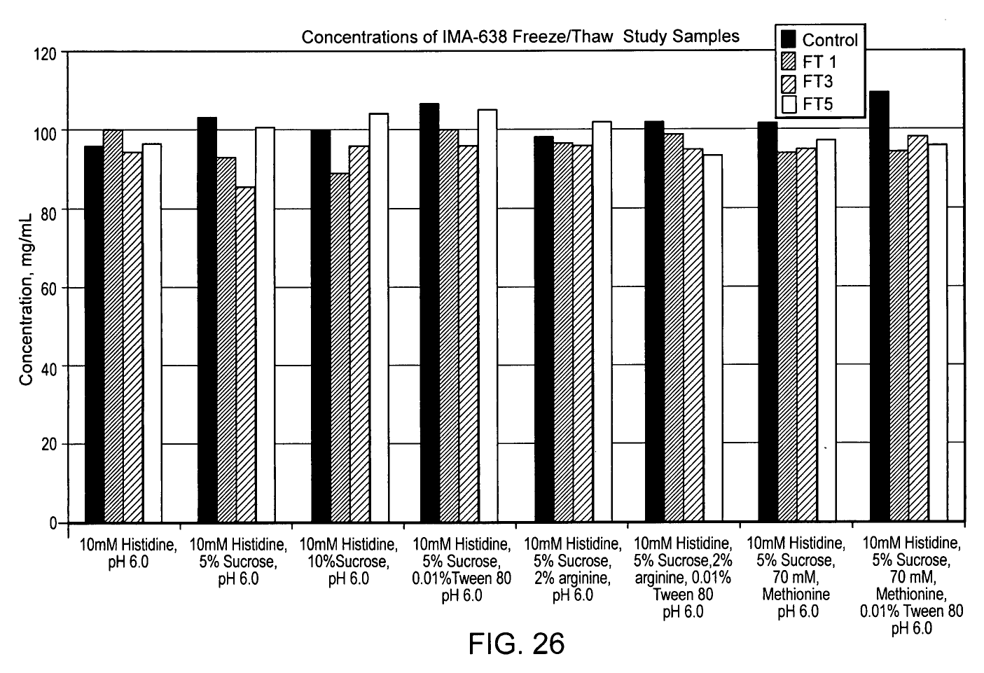

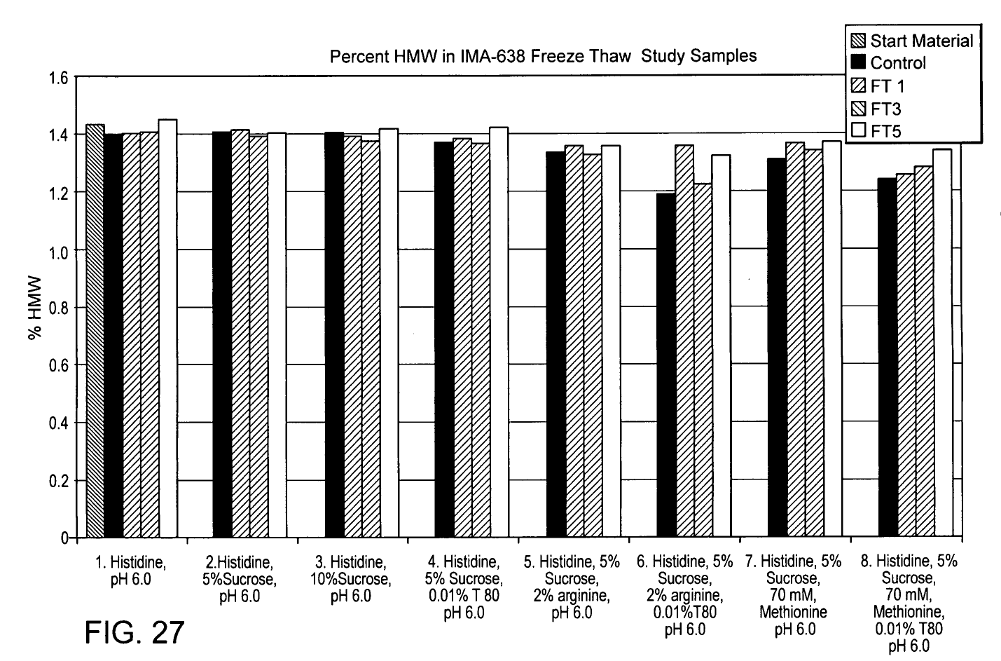

The freeze-thaw study was conducted by using 0.25 ml of 100 mg/ml IMA-638 liquid formulations in polypropylene tubes, wherein the freeze cycle was performed at −80° C. and the thaw cycle at 37° C. The freeze-thaw cycles were conducted once (FT1), thrice (FT3), or five times (FT5). The concentration of the samples following the freeze-thaw cycles compared to controls that were not subject to freeze-thaw cycles is shown in FIG. 26. The % HMW species following freeze-thaw was also determined and is shown in FIG. 27. The percent HMW species amongst the formulations following freeze-thaw ranged between about 1.2% to about 1.5%.

The presence of Tween did not demonstrate a clear effect on protecting against shear sensitivity with these conditions.

### Example 10

### Assessment of Liquid IL-13 Antibody Formulation in Pre-Filled Syringes

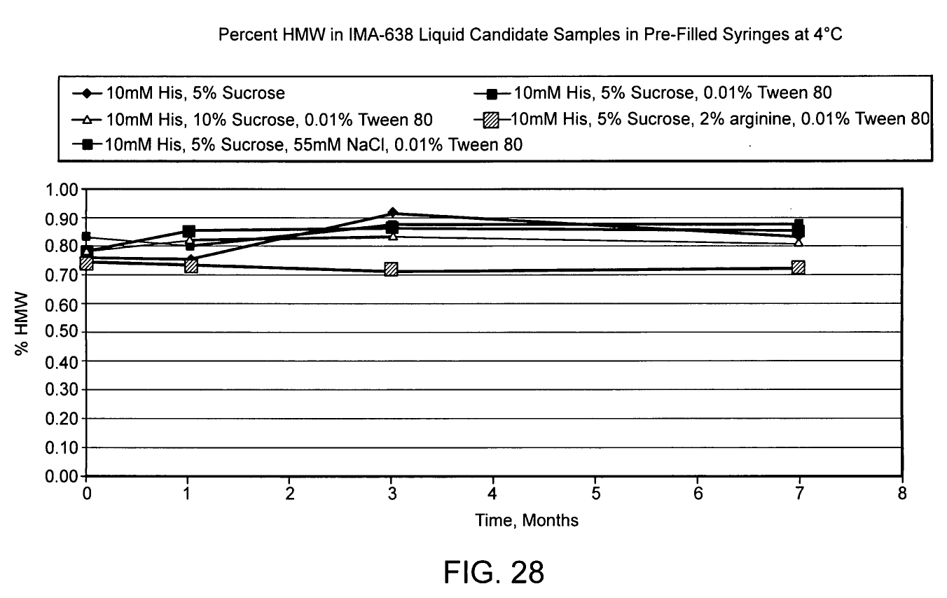

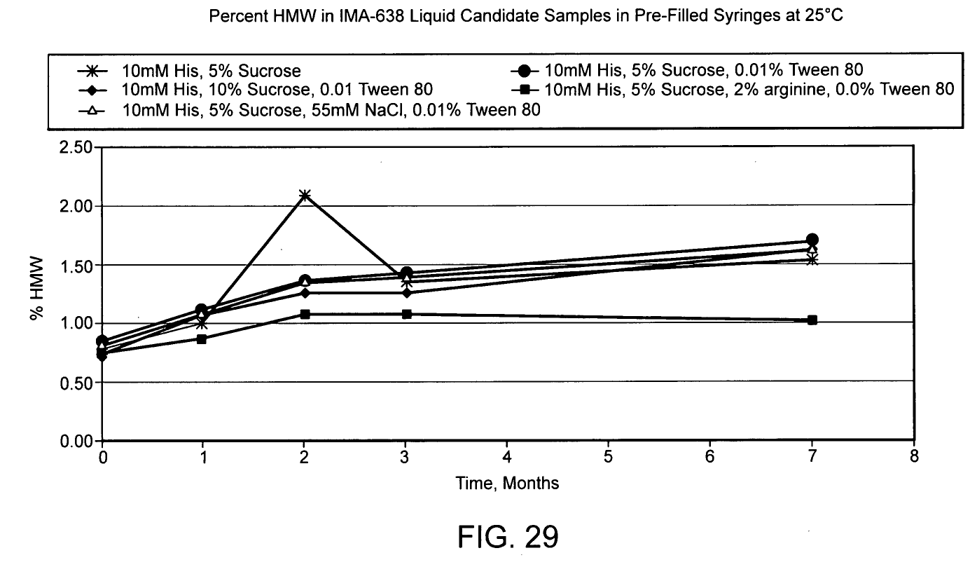

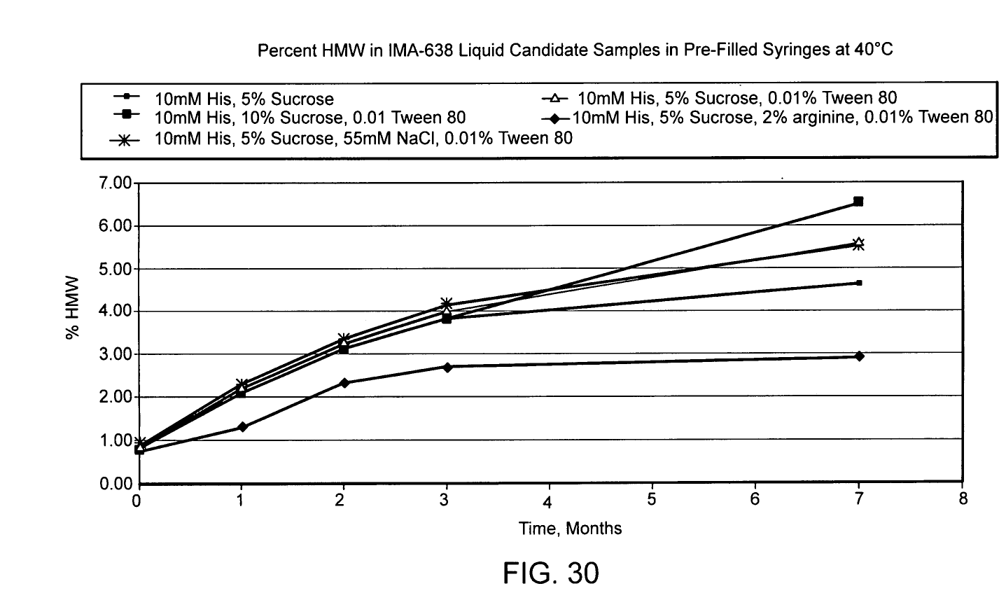

The stability of 100 mg/ml IMA-638 antibody formulations listed in Table 8 below packaged as 1 ml formulations in BD Hypak™ pre-filled syringes with West 4432/50 stoppers was assessed by determining the % HMW species at 4° C., 25° C., and 40° C. over seven months. The results of these studies are shown in FIGS. 28, 29, and 30.

TABLE 8
Hypak ™ Pre-Filled Syringe Formulations

| No. | Formulation |
|---|---|
| 1. | 10 mM Histidine, 5% Sucrose, pH 6.0 |
| 2. | 10 mM Histidine, 5% Sucrose, 0.01% Tween 80, pH 6.0 |
| 3. | 10 mM Histidine, 10% Sucrose, 0.01% Tween 80, pH 6.0 |
| 4. | 10 mM Histidine, 5% Sucrose, 2% Arginine, 0.01% Tween 80, |
| | pH 6.0 |
| 5. | 10 mM Histidine, 5% Sucrose, 55 mM NaCl, 0.01% Tween 80, |
| | pH 6.0 |

At 4° C. there were between 0.70% and 0.90% HMW species from t=0 months to t=seven months. At 25° C. there were between about 0.75% and about 2.00% HMW species, with the aggregates increasing over time. At 40° C. the aggregates increased in all formulations over time to between 4.5% to 6.5% at seven months for Formulations 1-3 and 5. The smallest increase in aggregates was observed for formulation 4 (about 3% at seven months).

The addition of arginine and Tween to a formulation consisting of 10 mM histidine and 5% sucrose appears to improve the stability of the IL-13 antibody in the context of pre-filled syringes at all temperatures studied.

Thus, one or both of these excipients could provide additional stability benefits to an anti-IL-13 formulation.

### Example 11

### Effect of Arginine on IMA-638 Liquid Formulations in Pre-Filled Syringes

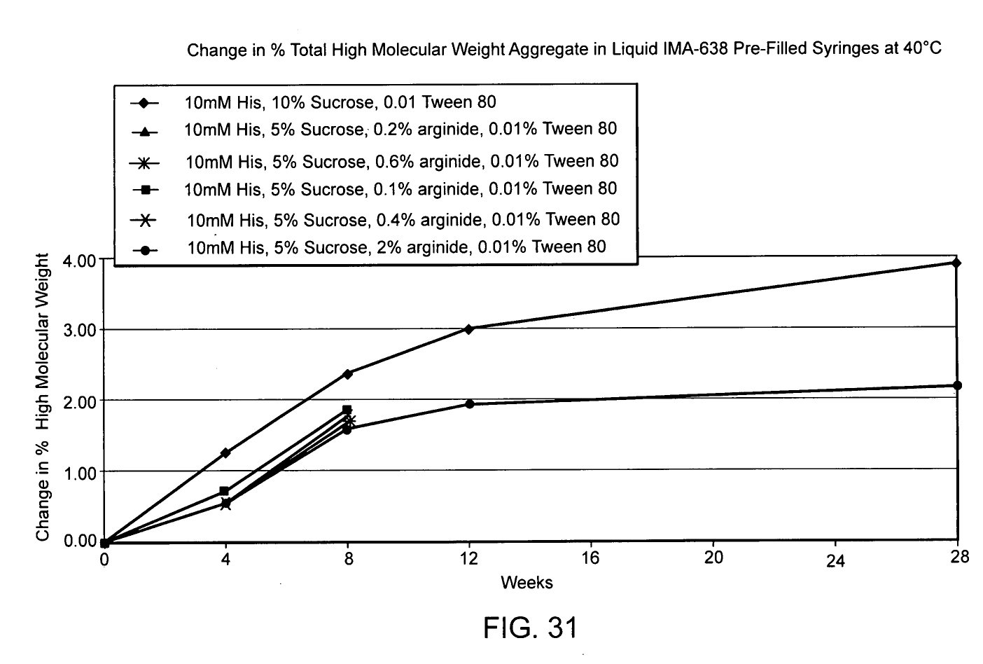

The effect of adding low concentrations of arginine (0.1%-2%) on the stability of 100 mg/ml IMA-638 antibody formulations formulated in 10 mM histidine, 5% sucrose, and 0.01% Tween 80 was studied by following the percent change in HMW species after four weeks, eight weeks, twelve weeks, and twenty-eight weeks of storage of pre-filled 1 ml BD Hypak™ SCF syringes with West W4023 Durafluor stoppers at 40° C. The results of this study are shown in FIG. 31.

The data indicates that the addition of arginine decreases the amount of HMW aggregation formed over time.

### Example 12

### Characterization of IMA-638 Aerosol from a PARI LC Plus Nebulizer

The IL-13 antibody formulations of the invention can be administered to a subject by a variety of means including as an aerosol. An aerosol is a suspension of liquid or solid particles in air. In some embodiments of the invention, the IL-13 antibody formulations are used for pulmonary delivery. The drug particles for pulmonary delivery are typically characterized by aerodynamic diameter rather than geometric diameter. The aerodynamic diameter is the diameter of a sphere of unit density (1 g/ml) that has the same gravitational settling velocity as the particle in question. Aerodynamic diameter takes into account physical properties that affect a particle's behavior in air such as density and shape. The velocity at which a particle settles is proportional to the aerodynamic diameter.

The median of the distribution of airborne particle mass with respect to the aerodynamic diameter is referred to as the mass median aerodynamic diameter (MMAD). The geometric standard deviation (GSD) is a measure of dispersion about the MMAD. Finally, the fine particle fraction (FPF) is the fraction of particles that are below a specified aerodynamic diameter (less than 4.7 Mm). The MMAD, GSD and FPF are measured by an Anderson Cascade Impactor (ACI). The ACI measures size distribution of droplets/particles generated from a nebulizer, a metered dose inhaler, a dry powder inhaler, the environment, etc.

In this experiment, the MMAD, GSD and FPF of aerosols produced from a 50 mg/ml and 0.5 mg/ml IMA-638 formulation (10 mM Histidine, 5% Sucrose, pH 6.0) from a PAR1 LC Plus Nebulizer were determined. Table 9 provides the results of this study.

TABLE 9

| | 50 mg/ml IMA-638 | 0.5 mg/ml IMA-638 |
|---|---|---|
| MMAD | 3.45 | 3.37 |
| GSD | 1.82 | 2.88 |
| FPF < 4.7 μm | 0.44 | 0.39 |

The IMA-638 formulations evaluated provide aerosol characteristics (including particle size and protein integrity) that are well suited for pulmonary delivery of anti-IL-13 antibodies by nebulization.

### Example 13

### Stability of the Lyophilized IL-13 Antibody, IMA-026

The long-term stability of a lyophilized anti-IL-13 antibody formulation was studied. Briefly, a formulation containing the anti-IL-13 antibody, IMA-026 (50 mg/ml), 10 mM histidine, 5% sucrose (weight/volume), pH 6.0, was prepared by sterile filtration and approximately 3.2 ml was dispensed into a 5 ml depyrogenated glass tubing vial having a West 4432/50 1319 siliconized gray stopper, and then lyophilized. The formulation was stored at 4° C., 25° C., or 40° C. for one month, two months, three months, six months, and twelve months at 4° C., 25° C., and 40° C., then the lyophilate was reconstituted using 1.3 ml sterile water (USP) to bring the reconstituted formulation to about 1.6 ml such that the formulation was 100 mg/ml anti-IL-13 antibody, 20 mM histidine, and 10% sucrose, pH 6.0.

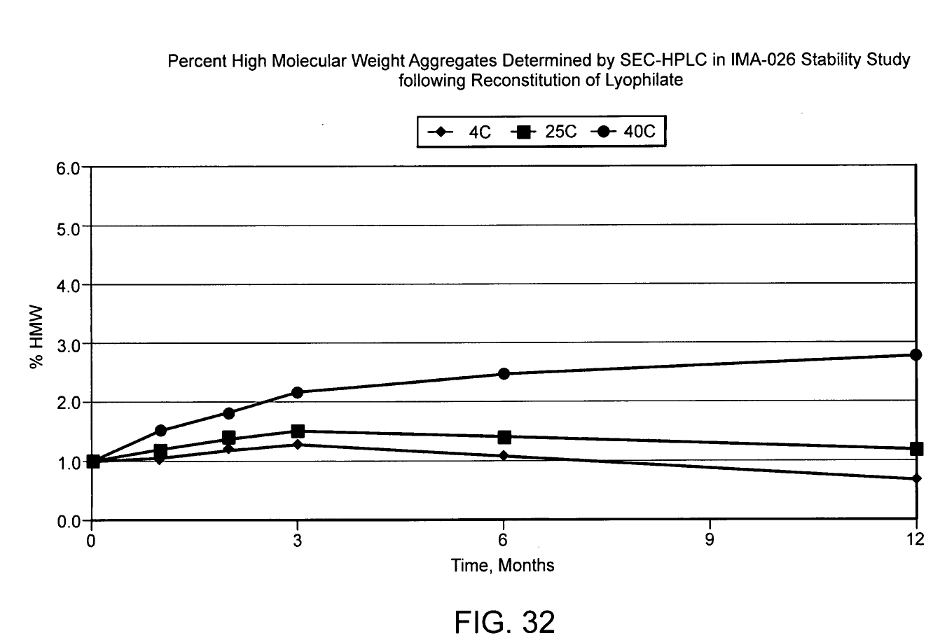

The percentage of HMW species was assayed using SEC-HPLC. The percentage of HMW species in the formulation before lyophilization and reconstitution was about 1% of the total protein in the formulation and was also about 1% in all samples stored at 4° C. and 25° C. (FIG. 32). After twelve months of storage at 40° C., the formulations were about 3.0% HMW species (FIG. 32). Thus, there was no substantial increase in the level of HMW species in samples stored at 5° C. and 25° C. for twelve months.

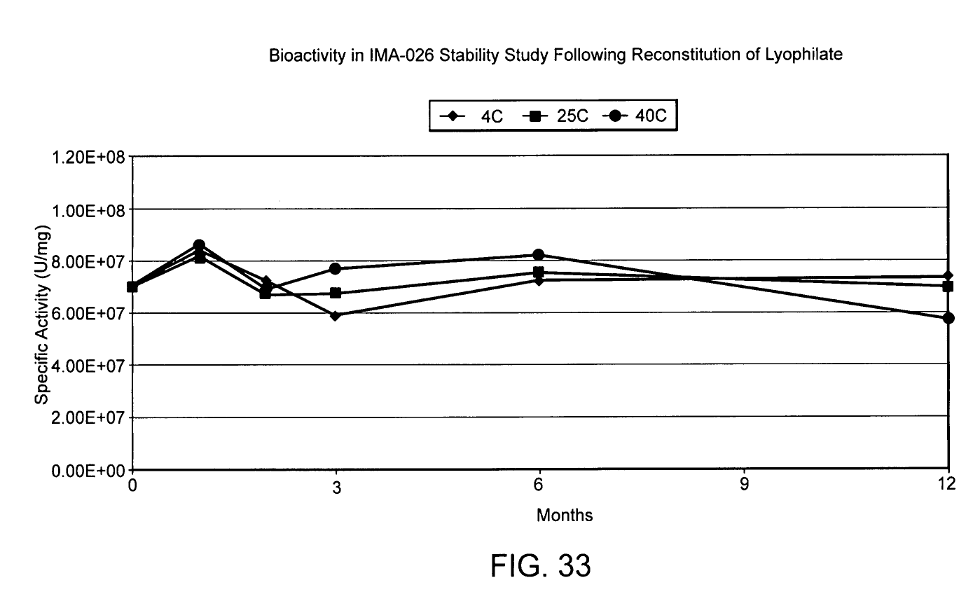

The lyophilized anti-IL-13 antibody formulations were also assayed for bioactivity using a cell-based assay in which inhibition of IL-13-dependent cell proliferation was examined in the presence of different concentrations of formulated antibody to demonstrate biological activity, i.e., the ability to bind and sequester IL-13 from the cells. The results of the assay are compared to the results using an anti-IL-13 antibody that was not stored. FIG. 33 illustrates the data from such a set of bioassays. Overall, there was no substantial change in the amount of bioactivity after twelve months of storage in any of the samples. Thus, the formulation is, as determined by bioactivity, suitable for storage of the lyophilized formulation for at least twelve months.

These data demonstrate that a lyophilized anti-IL-13 formulation as described herein is suitable for storage for at least twelve months.

### Example 14

### Stability of the Lyophilized IL-13 Antibody, IMA-026

This experiment was conducted as described in Example 1 except that the antibody used was IMA-026. The IMA-026 formulation used was: 50 mg/ml IMA-026, 10 mM Histidine, 5% Sucrose, 0.01% Tween-80, pH 6.0. The results were substantially similar to those obtained in Example 1. Thus, lyophilized IMA-026, like lyophilized IMA-638 is a stable formulation.

### Example 15

### Aerosolization of IMA-026 With and Without Tween

In this experiment, the effect of aerosolization of IMA-026 on % HMW, percent recovery, and bioactivity was studied. The data from this experiment are shown in Table 10 below.

TABLE 10

| | | | Bioactivity |
|---|---|---|---|
| | % HMW | % Recovery* | (U/mg) |
| Pre-nebulization | 0.13 | 100.0 | 6.40E+07 |
| Nebulized - | 0.13 | 76.0 | 7.30E+07 |
| no Tween | | | |
| Nebulized - | 0.14 | 81.3 | 6.08E+07 |
| w/Tween | | | |

*= by SEC-HPLC

As can be seen from Table 10, the pre and post nebulization properties of IMA-026 with or without Tween are substantially similar. Thus IMA-026 is suitable as an aerosol formulation.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An anti-IL-13 antibody formulation, comprising:
(a) an anti-IL-13 antibody;
(b) a cryoprotectant; and
(c) a buffer, wherein the pH of the formulation is about 5.5 to about 6.5.

2. The formulation of claim 1, wherein the formulation is a liquid formulation, a lyophilized formulation, a lyophilized formulation that is reconstituted as a liquid, or an aerosol formulation.

3. The formulation of claim 1, wherein the anti-IL-13 antibody in the formulation is at a concentration of: about 0.5 mg/ml to about 250 mg/ml, about 0.5 mg/ml to about 45 mg/ml, about 0.5 mg/ml to about 100 mg/ml, about 100 mg/ml to about 200 mg/ml, or about 50 mg/ml to about 250 mg/ml.

4. The formulation of claim 1, wherein the anti-IL-13 antibody is a humanized antibody.

5. The formulation of claim 4, wherein the antibody is a kappa light chain construct antibody.

6. The formulation of claim 4, wherein the antibody is selected from the group consisting of an IgG1 antibody, and IgG2 antibody, and an IgG4 antibody.

7. The formulation of claim 1, wherein the anti-IL-13 antibody is a monoclonal antibody.

8. The formulation of claim 1, wherein the anti-IL-13 antibody is IMA-638 or IMA-026.

9. The formulation of claim 1, wherein the cryoprotectant is about 2.5% to about 10% (weight/volume) sucrose or trehalose.

10. The formulation of claim 1, wherein the buffer is about 4 mM to about 60 mM histidine buffer, about 5 mM to about 25 mM succinate buffer, or about 5 mM to 25 mM acetate buffer.

11. The formulation of claim 1, wherein the formulation further comprises a surfactant at a concentration of about 0% to about 0.2%.

12. The formulation of claim 4, wherein the surfactant is selected from the group consisting of polysorbate-20, polysorbate-40, polysorbate-60, polysorbate-65, polysorbate-80, polysorbate-85, and combinations thereof.

13. The formulation of claim 1, wherein the formulation further comprises about 0.01% to about 5% arginine.

14. The formulation of claim 1, wherein the formulation further comprises about 0.001% to about 0.05% Tween.

15. The formulation of claim 1, wherein the formulation further comprises at least one of the following: about 1% to about 10% sorbitol, about 0.1% to about 2% glycine, about 5 mM to about 150 mM methionine, and about 5 mM to about 100 mM sodium chloride.

16. The formulation of claim 1, wherein the formulation further comprises a second antibody or an antigen-binding fragment thereof, wherein the second antibody is selected from the group consisting of: an anti-IL-13 antibody having a different epitope specificity than the IL-13 antibody of the formulation, an anti-IgE antibody, an anti-C5 antibody, an anti-IL-4 antibody, an anti-TNF-α antibody, and an anti-IL-9 antibody.

17. The formulation of claim 1, wherein the formulation further comprises a second therapeutically- or pharmacologically-active agent that is useful in treating an inflammatory disorder selected from the group consisting of an antihistamine, an anti-inflammatory agent, a long-acting bronchodilator (LABA), an inhaled corticosteroid (ICS), and a leukotriene inhibitor.

18. The formulation of claim 1, wherein
(a) the antibody is a humanized murine anti-IL-13 antibody;
(b) the cryoprotectant is about 0.02% to about 10% (weight/volume) sucrose or trehalose; and
(c) the buffer is about 4 mM to about 60 mM histidine buffer, pH 6.0.

19. The formulation of claim 18, wherein the formulation further comprises about 0.01% to about 5% arginine.

20. The formulation of claim 18, wherein the formulation further comprises about 0.001% to about 0.05% Tween.

21. The formulation of claim 18, wherein the formulation further comprises at least one of the following: about 1% to about 10% sorbitol, about 0.1% to about 2% glycine, about 5 mM to about 150 mM methionine, and about 5 mM to about 100 mM sodium chloride.

22. The formulation of claim 18, further comprising greater than 0% and up to about 0.2% polysorbate 80.

23. The formulation of claim 1, wherein
(a) the antibody is IMA-638 or IMA-026;
(b) the cryoprotectant is about 0.02% to about 10% (weight/volume) sucrose or trehalose; and
(c) the buffer is 10 mM succinate buffer, pH 6.0.

24. The formulation of claim 1, wherein
(a) the antibody is IMA-638 or IMA-026;
(b) the cryoprotectant is about 0.02% to about 10% (weight/volume) sucrose or trehalose; and
(c) the buffer is 10 mM acetate buffer, pH 6.0.

25. An aerosol formulation of an anti-IL-13 antibody, comprising:
(a) an anti-IL-13 antibody;
(b) about 5% to about 10% (weight/volume) sucrose or trehalose; and
(c) a buffer having a pH of about 5.5 to 6.5.

26. The formulation of claim 1, wherein the formulation further comprises about 0.01% to about 5% arginine.

27. The formulation of claim 1, wherein the formulation further comprises about 0.001% to about 0.05% Tween.

28. The formulation of claim 1, wherein the formulation further comprises at least one of the following: about 1% to about 10% sorbitol, about 0.1% to about 2% glycine, about 5 mM to about 150 mM methionine, and about 5 mM to about 100 mM sodium chloride.

29. The aerosol formulation of claim 25, further comprising a therapeutic agent that is useful in treating asthma or chronic obstructive pulmonary disease.

30. A lyophilized formulation of an anti-IL-13 antibody, comprising:
(a) an anti-IL-13 antibody;
(b) about 5% to about 10% (weight/volume) sucrose or trehalose; and
(c) a buffer having a pH of about 5.5 to 6.5.

31. The formulation of claim 1, wherein the percent increase in high molecular weight (HMW) species and low molecular weight (LMW) species compared to the original formulation is less than 5% after: at least eighteen months at −80° C., at least twenty-four months at −80° C., at least eighteen months at −20° C., at least twenty-four months at −20° C., at least eighteen months at 2° C.-8° C., at least twenty-four months at 2° C.-8° C., at least eighteen months at 25° C., or at least twenty-four months at 25° C.

32. The formulation of claim 31, wherein HMW and LMW species are assayed using size exclusion-high performance liquid chromatography (SEC-HPLC).

33. The formulation of claim 1, wherein at least 90% of the IL-13 antibody is monomeric antibody after storage of the antibody for at least eighteen months at 2° C.-8° C., or at least twenty-four months at 2° C.-8° C.

34. The formulation of claim 33, wherein the monomeric nature of the antibody is determined by a binding assay, a surface charge assay, a bioassay, or the ratio of HMW species to LMW species.

35. A pharmaceutical composition for the treatment of an IL-13-related disorder, the pharmaceutical composition comprising an anti-IL-13 antibody formulation of claim 1.

36. The pharmaceutical composition of claim 35, wherein the composition further comprises about 0.01% to about 5% arginine.

37. The pharmaceutical composition of claim 35, wherein the composition further comprises about 0.001% to about 0.05% Tween.

38. The pharmaceutical composition of claim 35, wherein the composition further comprises at least one of the following: about 1% to about 10% sorbitol, about 0.1% to about 2% glycine, about 5 mM to about 150 mM methionine, about mM to about 100 mM sodium chloride, and greater than 0% and up to about 0.2% of a surfactant.

39. The pharmaceutical composition of claim 35, wherein the composition comprises a humanized IL-13 antibody.

40. A manufacture of a pharmaceutical composition, the composition comprising an antibody formulation comprising:
(a) an anti-IL-13 antibody;
(b) a cryoprotectant; and
(c) a buffer, wherein the pH of the formulation is about 5.5 to 6.5.

41. A method of treating an IL-13-related disorder, the method comprising administering a pharmaceutically-effective amount of an antibody formulation comprising:
(a) an anti-IL-13 antibody;
(b) a cryoprotectant; and
(c) a buffer, wherein the pH of the formulation is about 5.5 to 6.5.

42. The method of claim 41, wherein the IL-13-related disorder is selected from the group consisting of: allergic asthma, non-allergic asthma, combinations of allergic and non-allergic asthma, exercise induced asthma, drug-induced asthma, occupational asthma, late stage asthma, chronic obstructive pulmonary disease, arthritis, inflammatory bowel disease, an inflammatory skin disorder, multiple sclerosis, osteoporosis, tendonitis, allergic disorders, inflammation in response to an insult to the host, sepsis, rheumatoid arthritis, osteoarthritis, irritable bowel disease, ulcerative colitis, psoriasis, systematic lupus erythematosus, an autoimmune disease, B-cell chronic lymphocytic leukemia (B-cell CLL), Hodgkin's disease, and tissue fibrosis in schistosomiasis.

43. The method of claim 41, wherein the antibody formulation is administered by a method selected from the group consisting of: oral, nasal, depot, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraocular, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, transdermal (topical), transmucosal, or sustained-release administration.

44. An injectable syringe containing a pre-filled solution of the formulation of claim 1.

45. A device for nasal administration comprising the formulation of claim 1 and a pharmaceutically-acceptable dispersant.

46. A transdermal patch comprising the formulation of claim 1 and optionally a pharmaceutically-acceptable carrier.

47. An intravenous bag comprising the formulation of claim 1 and optionally normal saline or 5% dextrose.

48. A kit comprising at least one container comprising the formulation of claim 1 and instructions for use.

49. The kit of claim 48, wherein the container is a glass vial or an injectable syringe.

50. A pre-filled injectable syringe, comprising the formulation:
(a) 100 mg/ml of an anti-IL-13 antibody;
(b) 10 mM histidine;
(c) 5% sucrose;
(d) 0.01% Tween-80;
(e) 40 mM NaCl,
wherein the pH of the formulation is 6.0.


SEQUENCE LISTING (16 sequences)

SEQ ID NO: 1 - protein, 448 aa, Artificial Sequence
  Description of Artificial Sequence Synthetic polypeptide
EVQLVESGGGLVQPGGSLRLSCAASGFTFISYAMSWVRQAPGKGLEWVASISSGGNTYYPDSVKGRFTISRDNAKNSLYLQMNSLRAEDTAVYYCARLDGYYFGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 218 aa, Artificial Sequence
  Description of Artificial Sequence Synthetic polypeptide
DIQMTQSPSSLSASVGDRVTITCKASESVDNYGKSLMHWYQQKPGKAPKLLIYRASNLESGVPSRFSGSGSGTDFTLTISSLQPEDFATYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 450 aa, Artificial Sequence
  Description of Artificial Sequence Synthetic polypeptide
EVQLVESGGGLVQPGGSLRLSCAASGFNIKDTYIHWVRQAPGKGLEWIGRIDPANDNIKYDPKFQGRFTISADNAKNSAYLQMNSLRAEDTAVYYCARSEENWYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 219 aa, Artificial Sequence
  Description of Artificial Sequence Synthetic polypeptide
DIQMTQSPSSLSASVGDRVTITCRSSQSIVHSNGNTYLEWYQQKPGKAPKLLIYKVSNRFSGVPSRFSGSGSGTDFTLTISSLQPEDFATYYCFQGSHIPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 5 aa, Artificial Sequence
  Description of Artificial Sequence Synthetic peptide
DTYIH

SEQ ID NO: 6 - protein, 17 aa, Artificial Sequence
  Description of Artificial Sequence Synthetic peptide
RIDPANDNIKYDPKFQG

SEQ ID NO: 7 - protein, 11 aa, Artificial Sequence
  Description of Artificial Sequence Synthetic peptide
SEENWYDFFDY

SEQ ID NO: 8 - protein, 8 aa, Artificial Sequence
  Description of Artificial Sequence Synthetic peptide
GFNIKDTY

SEQ ID NO: 9 - protein, 8 aa, Artificial Sequence
  Description of Artificial Sequence Synthetic peptide
IDPANDNI

SEQ ID NO: 10 - protein, 11 aa, Artificial Sequence
  Description of Artificial Sequence Synthetic peptide
SEENWYDFFDY

SEQ ID NO: 11 - protein, 16 aa, Artificial Sequence
  Description of Artificial Sequence Synthetic peptide
RSSQSIVHSNGNTYLE

SEQ ID NO: 12 - protein, 7 aa, Artificial Sequence
  Description of Artificial Sequence Synthetic peptide
KVSNRFS

SEQ ID NO: 13 - protein, 9 aa, Artificial Sequence
  Description of Artificial Sequence Synthetic peptide
FQGSHIPYT

SEQ ID NO: 14 - protein, 11 aa, Artificial Sequence
  Description of Artificial Sequence Synthetic peptide
QSIVHSNGNTY

SEQ ID NO: 15 - protein, 3 aa, Artificial Sequence
  Description of Artificial Sequence Synthetic peptide
KVS

SEQ ID NO: 16 - protein, 9 aa, Artificial Sequence
  Description of Artificial Sequence Synthetic peptide
FQGSHIPYT